(19) United States
(12) Patent Application Publication
(10) Pub. No.: US 2003/0203030 A1
(43) Pub. Date: Oct. 30, 2003

(54) Polymeric gel delivery system for pharmaceuticals

(75) Inventors: Paul Ashton, Boston, MA (US); Jianbing Chen, Belmont, MA (US)

Correspondence Address: ROPES & GRAY LLP, ONE INTERNATIONAL PLACE, BOSTON, MA, 02110-2624, US

(73) Assignee: Control Delivery Systems, Inc., Watertown, MA

(21) Appl. No.: 10/349,202

(22) Filed: Jan. 21, 2003

Related U.S. Application Data
(60) Provisional application No. 60/349,241, filed on Jan. 18, 2002.

(51) Int. Cl. A61K 38/19; A61K 31/192; A61K 31/60; A61K 9/14; A61K 31/56

(52) U.S. Cl. 424/484; 424/85.1; 514/179; 514/570; 514/165

(57) ABSTRACT

Implantable, injectable, insertable, or otherwise administrable compositions that form hydrogels when implanted, injected, inserted, or administered into or onto living tissues comprise a pharmaceutically effective compound wherein the pharmaceutically effective compound is a codrug, or pharmaceutically acceptable salt or prodrug thereof in admixture with a hydrogel-forming compound. The pharmaceutically effective compound may be any compound that is soluble in bodily fluids, or that forms bodily fluid-soluble adducts when exposed to bodily fluids. Exemplary compounds include analgesic, anti-inflammatory and antibiotic compounds. The hydrogel-forming compound is a biologically tolerated substance that forms a hydrogel upon exposure to bodily fluids, such as the interstitial fluid surrounding or within a joint.

### CROSS-REFERENCE TO RELATED APPLICATIONS

This application claims the benefit of priority from U.S. Provisional Application No. 60/349,241, filed Jan. 18, 2002, the specification of which is incorporated by reference herein in its entirety.

### FIELD OF THE INVENTION

The present invention relates to a novel drug delivery system. In particular, the present invention relates to an implantable, injectable, insertable, or otherwise administrable drug delivery composition that forms a hydrogel in a living tissue, and a method of using the composition to treat a living tissue in need of such treatment.

### BACKGROUND OF THE INVENTION

For a drug to be effective, a certain concentration must be maintained for a certain period of time at specific location(s). Systemically administered drugs may accomplish the first two objectives, but in an inefficient fashion and with the potential for toxic side effects. Local administration of controlled release formulations accomplishes all these objectives with a more efficient utilization of the drug and may reduce side effects.

However, local delivery of drug compounds to living tissue presents a number of problems, among them being the problem of effectively delivering drug to tissues in need of therapeutic treatment and the problem of in vivo instability of various potentially therapeutic agents. Certain therapeutic agents show remarkable promise in vitro, but are not stable in aqueous environments, such as are typical in vivo.

While it is possible to introduce certain therapeutic agents to specific loci in non-aqueous carriers, such as oils, such therapeutic methods suffer additional limitations. Several non-aqueous vehicles are not tolerated by all patient subpopulations. In fact some patients are especially sensitive to certain non-aqueous carriers such as peanut oil. Furthermore, the use of a non-aqueous liquid carrier does not solve the problem of delivery of drugs that are hydrophilic and relatively unstable in aqueous solution.

There remains a need for an improved injectable; implantable, insertable, or otherwise administrable drug delivery composition that provides release of a pharmaceutically active compound to a biological tissue in need of such treatment, wherein the composition is generally well-tolerated by the target patient population.

These and other needs are met by embodiments according to the present invention, as set forth herein.

### SUMMARY OF THE INVENTION

The present invention provides novel implantable, injectable, insertable, or otherwise administrable compositions for the treatment of a patient in need of delivery of one or more drug compounds to a biological tissue. The compositions according to the present invention comprise a codrug in admixture with a hydrogel-forming compound in vivo.

The present inventors have discovered that when a codrug is combined with a compound that forms a hydrogel in a living biological tissue, the resulting composition may be injected directly into or onto a living biological tissue without first forming the hydrogel prior to implantation, injection, insertion, or administration. The present inventors have found that when a codrug is combined with a hydrogel-forming compound, the resulting composition, which is substantially free of water, can be inserted, injected, or implanted into or onto a living tissue, such as a joint or the environs thereof, where the hydrogel-forming compound will swell with water from the surrounding living tissue as it forms a hydrogel. The inventors have also discovered a composition of a codrug combined with a hydrogel-forming compound that may also be hydrated prior to injection, implantation, insertion, or administration.

The release rate of a pharmaceutically acceptable compound may be adjusted by changing the codrug or hydrogel-forming compound used in the composition and/or by adjusting the porosity of the resultant hydrogel. The porosity of the hydrogel may be selected by adjusting the relative concentrations of the hydrogel-forming compound and the codrug. In this manner, the person skilled in the art can prepare biologically tolerated compositions that will gradually release pharmaceutically active compounds into or onto a living biological tissue over time. Alternatively, codrugs may be formulated with a hydrogel-forming compound such that release of a pharmaceutically active compound from the system is governed largely by the dissolution of the codrug within the hydrogel and not by diffusion of the pharmaceutically active compounds through the hydrogel. In such a system, the diffusion coefficient of a drug molecule or ion through the hydrogel is substantially the same as that through water. In yet other systems the hydrogel-forming compound may act to increase the rate of hydration of the drug delivery composition and increase the rate of drug release.

### BRIEF DESCRIPTION OF THE DRAWINGS

FIG. 1 is a graph of the release of triamcinolone acetonide (“TA”) and 5-fluorouracil (“5FU”) from a TA-5FU codrug/hyaluronic acid (“HA”) composition over time.

FIG. 2 is a graph of the release of ketorolac from a ketorolac-ketorolac codrug/HA composition over time.

FIG. 3 is a graph of the release of diclofenac from a diclofenac-diclofenac codrug/HA composition over time.

FIG. 4 is a graph of in vitro morphine release profiles for subcutaneous formulations.

FIG. 5 is a graph of in vitro morphine release profiles for intra-articular formulations.

### DETAILED DESCRIPTION OF THE INVENTION

One aspect of the invention provides a pharmaceutical composition comprising a codrug, or a pharmaceutically acceptable salt or prodrug thereof, in admixture with a hydrogel-forming compound, wherein the codrug comprises:

a) at least two constituent moieties, each moiety being a residue of a biologically active compound or a prodrug thereof, including a first constituent moiety and a second constituent moiety; and

b) a linkage covalently linking the at least two constituent moieties to form the codrug,

wherein the linkage is cleaved under physiological conditions to regenerate the constituent moieties.

In some embodiments, the first constituent moiety is selected from analgesic compounds, anti-inflammatory steroidal compounds (corticosteroids), non-steroidal anti-inflammatory compounds (NSAIDs), antibiotic compounds, anti-fungal compounds, antiviral compounds, antiproliferative compounds, antiglaucoma compounds, immunomodulatory compounds, cell transport/mobility impeding agents, cytokines and peptides/proteins, alpha-blockers, anti-androgens, anti-cholinergic, adrenergic, purinergic, dopaminergic, local anesthetics, vanilloids, anti-angiogenic agents, nitrous oxide inhibitors, anti-apoptotic agents, macrophage activation inhibitors, and antimetabolite compounds.

In certain embodiments, the second constituent moiety is selected from analgesic compounds, anti-inflammatory steroidal compounds (corticosteroids), non-steroidal anti-inflammatory compounds (NSAIDs), antibiotic compounds, anti-fungal compounds, antiviral compounds, antiproliferative compounds, antiglaucoma compounds, immunomodulatory compounds, cell transport/mobility impeding agents, cytokines and peptides/proteins, alpha-blockers, anti-androgens, anti-cholinergic, adrenergic, purinergic, dopaminergic, local anesthetics, vanilloids, anti-angiogenic agents, nitrous oxide inhibitors, anti-apoptotic agents, macrophage activation inhibitors, and antimetabolite compounds.

In some embodiments, the codrug has the following structural formula:

R₁-L-(R₂)ₙ

wherein the first constituent moiety is R₁;

the second constituent moiety is R₂;

R₁ and R₂ each represent, independently, a residue of a compound selected from analgesic compounds, anti-inflammatory steroidal compounds (corticosteroids), non-steroidal anti-inflammatory compounds (NSAIDs), antibiotic compounds, anti-fungal compounds, antiviral compounds, antiproliferative compounds, antiglaucoma compounds, immunomodulatory compounds, cell transport/mobility impeding agents, cytokines and peptides/proteins, alpha-blockers, anti-androgens, anti-cholinergic, adrenergic, purinergic, dopaminergic, local anesthetics, vanilloids, anti-angiogenic agents, nitrous oxide inhibitors, anti-apoptotic agents, macrophage activation inhibitors, and antimetabolite compounds;

n is an integer of from 1 to 4; and

L is selected from a direct bond and a linking group.

In other embodiments, the codrug has the following structural formula:

R₁-(L-R₂)n

wherein the first constituent moiety is R₁;

the second constituent moiety is R₂;

R₁ and R₂ each represent, independently, a residue of a compound selected from analgesic compounds, anti-inflammatory steroidal compounds (corticosteroids), non-steroidal anti-inflammatory compounds (NSAIDs), antibiotic compounds, anti-fungal compounds, antiviral compounds, antiproliferative compounds, antiglaucoma compounds, immunomodulatory compounds, cell transport/mobility impeding agents, cytokines and peptides/proteins, alpha-blockers, anti-androgens, anti-cholinergic, adrenergic, purinergic, dopaminergic, local anesthetics, vanilloids, anti-angiogenic agents, nitrous oxide inhibitors, anti-apoptotic agents, macrophage activation inhibitors, and antimetabolite compounds;

n is an integer of from 1 to 4; and

L is selected from a direct bond and a linking group.

In yet other embodiments, the codrug has the following structural formula:

(R₁-L)ₘR₂(L₂-R₃)ₙ

wherein the first constituent moiety is R₁;

the second constituent moiety is R₂;

the third constituent moiety is R₃;

R₁, R₂, and R₃ each represent, independently, a residue of a compound selected from analgesic compounds, anti-inflammatory steroidal compounds (corticosteroids), non-steroidal anti-inflammatory compounds (NSAIDs), antibiotic compounds, anti-fungal compounds, antiviral compounds, antiproliferative compounds, antiglaucoma compounds, immunomodulatory compounds, cell transport/mobility impeding agents, cytokines and peptides/proteins, alpha-blockers, anti-androgens, anti-cholinergic, adrenergic, purinergic, dopaminergic, local anesthetics, vanilloids, anti-angiogenic agents, nitrous oxide inhibitors, anti-apoptotic agents, macrophage activation inhibitors, and antimetabolite compounds;

m is an integer of from 1 to 4;

n is an integer of from 1 to 4; and

L and L₂ are each independently selected from a direct bond and a linking group.

In some embodiments, R₁ and/or R₂ is a residue of diclofenac, etodolac, ketorolac, indomethacin, salicylic acid, sulindac, tolmetin, nabumetone, piroxicam, acetaminophen, fenoprofen, flurbiprofen, ibuprofen, ketoprofen, naproxen, oxaprozin, aspirin, choline magnesium trisalicylate, diflunisal, meclofenamic acid, mefenamic acid, phenylbutazone, or analog, derivative, or salt thereof.

In other embodiments, R₁ is a residue of alitretinoin (9-cis-retinoic acid); amifostine; bexarotene (4-[1-(5,6,7,8-tetrahydro-3,5,5,8,8-pentamethyl-2-naphthalenyl) ethenyl] benzoic acid); bleomycin; capecitabine (5′-deoxy-5-fluoro-cytidine); chlorambucil; bleomycin; BCNU; cladribine; cytarabine; daunorubicin; docetaxel; doxorubicin; epirubicin; estramustine; etoposide; exemestane (6-methylenandrosta-1,4-diene-3,17-dione); fludarabine; 5-fluorouracil; gemcitabine; hydroxyurea; idarubicin; irinotecan; melphalan; methotrexate; mitoxantrone; paclitaxel; pentostatin; streptozocin; temozolamide; teniposide; tomudex; topotecan; valrubicin (N-trifluoroacetyladriamycin-14-valerate); vinorelbine; or analog, derivative, or salt thereof.

In certain embodiments, R₂ is a residue of:

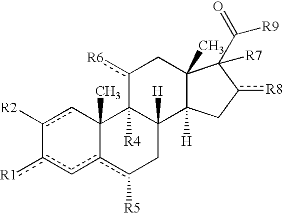

wherein R1 is ═O, —OH, or —(CH₂)₁₋₄Cl;

R2 is H, C₁₋₄alkyl, Cl, or Br;

R4 is H, F, or Cl;

R5 is H, F, Cl, CH₃, or —CHO;

R6 is H, OH, or Cl;

R7 is H, OH, CH₃, O—COCH₃, O(CO)OCH₂CH₃, O—(CO)-2-furanyl, or O—C(O)—(CH₂)₂CH₃;

R8 is H, CH₃, OH, ═CH₂, or together R7 and R8 form, together with the adjacent carbon atoms to which they are attached:

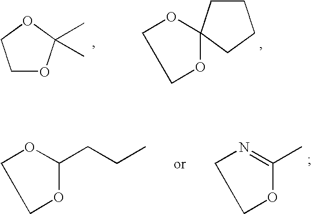

and

R9 is CH₃, CH₂OH, CH₂O(CO)CH₃, CH₂—O—C₁₋₄alkyl, CH₂Cl, —OCH₂Cl, —CH₂—N—(N′-methyl)piperazinyl, —CH₂—O—(CO)—CH₂—N(Et)₂, ethyl, CH₂SH, CH₂O(CO)C₁₋₄alkyl, CH₂(CO)C(2-propyl)—NH(CO)C₆H₅, or —S—CH₂—F; and

wherein the bonds indicated by [symbol] are either double or single bonds.

In some embodiments, R₂ is a residue of 21-acetoxypregnenolone, alclometasone, algestone, amcinonide, beclomethasone, betamethasone, budesonide, chloroprednisone, clobetasol, clocortolone, cloprednol, corticosterone, cortisone, cortivazol, deflazacort, desonide, desoximetasone, dexamethasone, diflorasone, diflucortolone, difuprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, hydrocortisone, loteprednol etabonate, mazipredone, medrysone, meprednisone, methylprednisolone, mometasone furoate, paramethasone, prednicarbate, prednisolone, prednisolone 25-diethylaminoacetate, prednisolone sodium phosphate, prednisone, prednival, prednylidene, rimexolone, tixocortol, triamcinolone, triamcinolone acetonide, triamcinolone benetonide, triamcinolone hexacetonide, or analog, derivative, or salt thereof.

In certain embodiments, the first constituent moiety is the same as the second constituent moiety. In other embodiments, the first constituent moiety is different from the second constituent moiety.

In some embodiments, the pharmaceutical composition comprises less than 15 wt. % water. In other embodiments, the pharmaceutical composition contains less than 10 wt. % water, or less than about 5 wt. % water.

In certain embodiments, the pharmaceutical composition comprises from about 5 wt. % to about 90 wt. % codrug. In some embodiments, the pharmaceutical composition comprises from about 30 wt. % to about 80 wt. % codrug, more preferably from about 50 wt. % to about 70 wt. % codrug.

In some embodiments, the hydrogel-forming compound forms a physical gel. In certain embodiments, the hydrogel-forming compound is hyaluronic acid or a derivative thereof. In some embodiments, the hydrogel-forming compound forms a chemical gel.

In some embodiments, the pharmaceutical composition is hydrated prior to implantation, injection, insertion, or administration.

In some embodiments, the composition is in the form of an implantable, injectable, insertable, or otherwise administrable pellet, tablet, caplet, or capsule. In certain embodiments, the composition is in the form of an implantable, injectable, insertable, or otherwise administrable pellet.

In some embodiments, the pellet has a diameter from about 0.1 mm to about 5.0 mm, preferably from about 0.5 mm to about 2.4 mm, more preferably from about 0.8 mm to about 2.0 mm. In some embodiments, the pellet has a length of from about 0.3 mm to about 3.0 mm, preferably from about 0.3 mm to about 2.5 mm, more preferably from about 0.7 mm to about 2 mm. In certain embodiments, the pellet is sized for administration with standard-sized needles, for example, a 16 or 18 gauge needle.

In some embodiments, the pellet weighs from about 0.5 g to about 5.0 g, preferably from about 1.0 g to about 2.0 g.

In certain embodiments, the pharmaceutical composition further comprises a pharmaceutically acceptable carrier, excipient, solvent, adjuvant, additive, diluent, dispersant, or surfactant. In some embodiments, the pharmaceutically acceptable carrier comprises a biocompatible polymer. In some embodiments, the polymer is selected from collagen, carbopol, hydroxypropylmethyl cellulose (“HPMC”), polyanhydride, polylactic acid, poly(ethylene glycol) (“PEG”), and poly(ethylene-co-vinyl acetate). In certain embodiments, the pharmaceutically acceptable additive is selected from sodium alginate, magnesium stearate, and CaHPO₄.

In some embodiments, the pharmaceutical composition is in an implantable, injectable, insertable, or otherwise administrable single-dosage form. In some embodiments, the pharmaceutical composition is in an implantable, injectable, insertable, or otherwise administrable partial-dosage form. In certain embodiments, more than one partial-dosage form is implanted, injected, inserted, or administered to provide a therapeutically effective amount of at least one constituent moiety of a codrug. A single-dosage or partial-dosage form may be in the form of an implantable, injectable, insertable, or otherwise administrable pellet, tablet, caplet, or capsule. The number and size of pellets, tablets, caplets, or capsules administered will depend on a variety of factors such as the amount of codrug included in each unit, the therapeutically effective amount of at least one constituent moiety of a codrug, the disease, disorder, or condition to be treated, the joint or tissue to be treated, etc.

In some embodiments, from about 5 to about 40 units are administered into or onto a joint or tissue, more preferably from about 10 to about 30 units.

In some embodiments, the pharmaceutical composition when placed in the body hydrates to release drug such that the rate of release of the drug is controlled by the dissolution of the codrug within the hydrogel. In certain embodiments, the pharmaceutical composition hydrates when placed in the body and releases drug such that a diffusion coefficient of drug molecules or ions through the hydrogel is substantially the same as the diffusion coefficient of drug molecules or ions through a surrounding bodily fluid.

In some embodiments, the hydrogel-forming compound disperses before about 30% to about 50% of the codrug in the composition is released.

In certain embodiments, the first and second constituent moieties are directly linked through a covalent bond formed between a functional group of the first constituent moiety and a functional group of the second constituent moiety. In other embodiments, the first and second constituent moieties are linked to one another via a linking group that is covalently bonded to the first and second constituent moieties via functional groups thereon.

In certain embodiments, the first constituent moiety is an NSAID compound. In some embodiments, the second constituent moiety is an analgesic compound. In certain embodiments, the first constituent moiety is diclofenac or ketorolac and the second constituent moiety is morphine.

In certain embodiments, the first constituent moiety is an antiproliferative agent and the second constituent moiety is an NSAID, with the proviso that the first constituent moiety is not floxuridine, and with the further proviso that when the first constituent moiety is 5-fluorouracil, the second constituent moiety is not flurbiprofen or indomethacin.

In some embodiments, the first constituent moiety is an antiproliferative agent and the second constituent moiety is a corticosteroid agent, with the proviso that when the antiproliferative agent is 5-fluorouracil, the corticosteroid is not fluocinolone acetonide, triamcinolone, triamcinolone acetonide, desoximetasone, or hydrocortisone-17-butyrate, and with the further proviso that the antiproliferative agent is not a 1-β-arabinofuranosylcytosine derivative.

In certain embodiments, a codrug, or a pharmaceutically acceptable salt or prodrug thereof, is distributed as particles within a hydrogel-forming compound.

In other embodiments, a codrug, or a pharmaceutically acceptable salt or prodrug thereof, is dissolved in a hydrogel-forming compound.

Another aspect of the invention provides a method of treatment, comprising administering to a patient in need thereof a therapeutically effective amount of at least one constituent moiety in a composition comprising a codrug, or a pharmaceutically acceptable salt or prodrug thereof, in admixture with a hydrogel-forming compound, wherein the codrug comprises:

a) at least two constituent moieties, each moiety being a residue of a biologically active compound or a prodrug thereof, including a first constituent moiety and a second constituent moiety; and

b) a linkage covalently linking the at least two constituent moieties to form the codrug,

wherein the linkage is cleaved under physiological conditions to regenerate the constituent moieties.

In some embodiments, the therapeutically effective amount is an amount effective to produce an analgesic, an anti-inflammatory, an antibiotic, an anti-fungal, an antiviral, and/or an antiproliferative effect in the patient.

In certain embodiments, the method of administering a pharmaceutical composition of the invention comprises implanting the codrug formulation into a synovial joint, a fibrous joint, or a cartilaginous joint, or the tissues surrounding said joint. In other embodiments, the method of administering a pharmaceutical composition of the invention comprises injecting the codrug formulation into a synovial joint, or the tissues surrounding said joint. In some embodiments, the method of administering a pharmaceutical composition of the invention comprises inserting the codrug formulation into a synovial joint, a fibrous joint, or a cartilaginous joint, or the tissues surrounding said joint. In some embodiments, the synovial joint is of a jaw, shoulder, knee, elbow, hip, ankle, wrist, finger, or toe. In some embodiments, the fibrous joint is a tooth, the alveoli, or the distal tibiofibular joint. In some embodiments, the cartilaginous joint is a vertebral disk. In some embodiments, the method of administering a pharmaceutical composition of the invention comprises implanting, injecting, or inserting the codrug formulation into the bursae or tendon sheath.

In some embodiments, the method of administering a biologically active agent to a patient, comprises implanting, injecting, or inserting a pharmaceutical composition comprising a codrug, or a pharmaceutically acceptable salt or prodrug thereof, in admixture with a hydrogel-forming compound, for administration of at least one biologically active moiety, which codrug comprises:

a) at least two constituent moieties, each moiety being a residue of a biologically active compound or a prodrug thereof, including a first constituent moiety and a second constituent moiety; and

b) a linkage covalently linking said at least two constituent moieties to form said codrug, wherein said linkage is cleaved under physiological conditions to regenerate said constituent moieties;

wherein the composition is implanted in a synovial joint, a fibrous joint, or a cartilaginous joint, or the tissues surrounding said joint.

In certain embodiments, the method of inhibiting cell proliferation in a patient in need of treatment comprises implanting, injecting, or inserting a pharmaceutical composition comprising a codrug, or a pharmaceutically acceptable salt or prodrug thereof, in admixture with a hydrogel-forming compound, for administration of at least one biologically active moiety, which codrug comprises:

a) at least two constituent moieties, each moiety being a residue of a biologically active compound or a prodrug thereof, including a first constituent moiety and a second constituent moiety; and

b) a linkage covalently linking said at least two constituent moieties to form said codrug, wherein said linkage is cleaved under physiological conditions to regenerate said constituent moieties;

wherein the composition includes a therapeutically effective amount of at least one constituent moiety of a codrug, or a pharmaceutically acceptable salt thereof.

In some embodiments, the method of inhibiting inflammation in a patient in need of treatment comprises implanting a pharmaceutical composition comprising a codrug, or a pharmaceutically acceptable salt or prodrug thereof, in admixture with a hydrogel-forming compound, for administration of at least one biologically active moiety, which codrug comprises:

a) at least two constituent moieties, each moiety being a residue of a biologically active compound or a prodrug thereof, including a first constituent moiety and a second constituent moiety; and

b) a linkage covalently linking said at least two constituent moieties to form said codrug, wherein said linkage is cleaved under physiological conditions to regenerate said constituent moieties;

wherein the composition includes a therapeutically effective amount of at least one constituent moiety of a codrug, or a pharmaceutically acceptable salt thereof.

In some embodiments, the method comprises implanting, injecting, or inserting a pharmaceutical composition of the invention into a synovial joint, a fibrous joint, or a cartilaginous joint, or the tissues surrounding the aforementioned joint.

In certain embodiments, the patient is being treated for an autoimmune disease, pain, or inflammation. In some embodiments, the autoimmune disease is rheumatoid arthritis.

Yet another aspect of the invention provides a method of manufacturing a pharmaceutical composition, comprising providing a codrug, or a pharmaceutically acceptable salt or prodrug thereof, wherein the codrug comprises:

a) at least two constituent moieties, each moiety being a residue of a biologically active compound or a prodrug thereof, including a first constituent moiety and a second constituent moiety; and

b) a linkage covalently linking said at least two constituent moieties to form said codrug, said linkage is cleaved under physiological conditions to regenerate said constituent moieties;

and combining the codrug with a hydrogel-forming compound.

In certain embodiments, the method of preparing a pharmaceutical composition according to the invention comprises combining a powder, including a codrug, with a hydrogel-forming compound.

In some embodiments, at least one constituent moiety of the codrug, taken alone, is effective for treating an autoimmune disease. In certain embodiments, at least one constituent moiety of the codrug, taken alone, is effective for treating rheumatoid arthritis or osteoarthritis. In certain embodiments, at least one constituent moiety of the codrug, taken alone, is effective for treating pain. In certain embodiments, at least one constituent moiety of the codrug, taken alone, is effective for treating inflammation.

In certain embodiments, the constituent moieties are steroids.

In some embodiments, the first constituent moiety is morphine. In certain embodiments, the second constituent is vitamin E or ethacrynic acid.

In some embodiments, the pharmaceutical composition further comprises a biocompatible polymer. In some embodiments, the biocompatible polymer is poly(ethylene glycol).

In some embodiments, the pharmaceutical composition comprises more than one hydrogel-forming compound. In some embodiments, the pharmaceutical composition comprises more than one polymer.

Still another aspect of the invention provides a pharmaceutical composition comprising a codrug of diclofenac covalently linked to morphine, hyaluronic acid, and poly(ethylene glycol).

In certain embodiments, the codrug comprises from about 5 wt. % to about 90 wt. % of the pharmaceutical composition, the hyaluronic acid or a derivative thereof comprises from about 10 wt. % to about 90 wt. % of the pharmaceutical composition, and the biocompatible polymer comprises from about 0 wt. % to about 50 wt. % of the pharmaceutical composition.

A yet further aspect of the invention provides an injectable pellet comprising a pharmaceutical composition according to the invention, wherein the pellet forms a hydrogel in vivo.

Still another aspect of the invention provides a kit comprising a pharmaceutical composition according to the invention, in association with instructions (written and/or pictorial) describing the use of the composition for treatment or prevention of autoimmune disease, pain, or inflammation and optionally, warnings of possible side effects and drug-drug interactions.

In preferred embodiments, the hydrogel-forming compound is hyaluronic acid (“HA”) having an average molecular weight of about of 5.0×10⁵ Daltons; more preferably a molecular weight between 1.5×10⁵ and 3×10⁶ Daltons; even more preferably between 3×10⁵ and 2.6×10⁶ Daltons; and most preferably the molecular weight of the HA is between 3.5×10⁵ and 1×10⁶ Daltons. As used herein, the term “HA” means hyaluronic acid and any of its hyaluronate salts. Preferably, the HA used in the composition of the invention is sodium-hyaluronate.

In certain embodiments, the pharmaceutical compositions of the present invention are administered prior to surgery, during surgery, or after surgery. In some embodiments, the pharmaceutical compositions are administered from between 1 to 5 days prior to surgery or after surgery. In some embodiments, the surgery includes arthroscopy, endoscopy, or laparascopy, etc. In certain embodiments, pharmaceutical compositions is administered through the channel of the arthroscope, endoscope, or laparascope.

I. Definitions

The term “ED₅₀” means the concentration of a drug that produces 50% of its maximum response or effect.

The term “IC₅₀” means the dose of a drug that inhibits a biological activity by 50%.

The term “LD₅₀” means the dose of a drug that is lethal in 50% of test subjects.

The term “therapeutic index” refers to the therapeutic index of a drug defined as LD₅₀/ED₅₀.

The term “active” as used herein means therapeutically or pharmacologically active.

An “autoimmune” disease is understood to be one where the target of the disease is “self” or “self antigen.” There are a number of diseases that are believed to involve T cell immunity directed to self antigens, including (but not limited to) multiple sclerosis (MS), Type I diabetes, and rheumatoid arthritis (RA). Other autoimmune diseases include but are not limited to Wegener's granulomatosis, Crohn's disease and systemic lupus erythematosus (lupus).

A “biocompatible” substance, as the term is used herein, is one that has no medically unacceptable toxic or injurious effects on biological function.

The term “biological tissue” means any tissue in a living organism. The term includes soft tissues, such as muscle, tendons, bursae, ligaments, connective tissues, bone marrow, abdominal organ tissues, etc., as well as skeletal tissue, such as bone and cartilage. In one embodiment according to the present invention, the biological tissue is a synovial joint, such as a jaw, toe, finger, knee, elbow, shoulder, hip, or wrist joint.

As used herein, the term “codrug” means a first constituent moiety chemically linked to at least one other constituent moiety that is the same as, or different from, the first constituent moiety. The individual constituent moieties are reconstituted as the pharmaceutically active forms of the same moieties, or codrugs thereof, prior to conjugation. Constituent moieties may be linked together via reversible covalent bonds such as ester, amide, carbamate, carbonate, cyclic ketal, thioester, thioamide, thiocarbamate, thiocarbonate, xanthate and phosphate ester bonds, so that at the required site in the body they are cleaved to regenerate the active forms of the drug compounds.

As used herein, the term “constituent moiety” means one of two or more pharmaceutically active moieties so linked as to form a codrug according to the present invention as described herein. In some embodiments according to the present invention, two molecules of the same constituent moiety are combined to form a dimer (which may or may not have a plane of symmetry). In the context where the free, unconjugated form of the moiety is referred to, the term “constituent moiety” means a pharmaceutically active moiety, either before it is combined with another pharmaceutically active moiety to form a codrug, or after the codrug has been hydrolyzed to remove the linkage between the two or more constituent moieties. In such cases, the constituent moieties are chemically the same as the pharmaceutically active forms of the same moieties, or codrugs thereof, prior to conjugation.

The term “drug,” refers to a pharmaceutically active compound for treatment of a biological tissue in need of therapeutic treatment. In some embodiments according to the present invention, suitable drugs include water-soluble pharmaceuticals, water-labile pharmaceuticals, and pharmaceuticals that are both water-soluble and water-labile.

The term “hydrogel-forming compound” as used herein, refers to materials that absorb solvents (such as water), undergo rapid swelling without discernible dissolution, and maintain three-dimensional networks capable of reversible deformation. The term “hydrogel-forming compound” applies both to its hydrated and unhydrated forms, e.g., before and after a compound achieves the characteristics of a hydrogel. Hydrogel-forming compounds may be uncrosslinked or crosslinked. Uncrosslinked hydrogel-forming compounds are able to absorb water but do not dissolve due to the presence of hydrophobic and hydrophilic regions. Covalently crosslinked networks of hydrophilic polymers, including water soluble polymers, are traditionally denoted as hydrogels in the hydrated state. A number of aqueous hydrogels have been used in various biomedical applications, such as, for example, soft contact lenses, wound management, and drug delivery. The synthesis, characterization, and the formation of hydrogels is described, e.g., in Sawhney et al., “Bioerodible Hydrogels Based on Photopolymerized Poly(ethyleneglycol)-co-poly(a-hydroxy acid) Diacrylate Macromers”, Macromolecules, 26:581-587 (1993).

Hydrogels can be formed from natural polymers such as glycosaminoglycans, polysaccharides, and proteins. Hydrophilic polymeric materials suitable for use in forming hydrogels include poly(hydroxyalkylmethacrylate), poly(electrolyte complexes), poly(vinylacetate) cross-linked with hydrolysable bonds, water-swellable N-vinyl lactams polysaccharides, natural gum, agar, agarose, sodium alginate, carrageenan, fucoidan, furcellaran, laminaran, hypnea, eucheuma, gum arabic, gum ghatti, gum karaya, gumtragacanth, locust beam gum, arabinogalactan, pectin, amylopectin, gelatin, carboxymethyl cellulose, ethylcellulose, methylcellulose, hydropropyl methyl cellulose, hydrophilic colloids such as carboxymethyl cellulose gum or alginate gum cross-linked with a polyol such as propylene glycol, and salts and derivatives thereof. Several formulations of previously known hydrogels are described in U.S. Pat. Nos. 3,640,741 to Etes, 3,865,108 to Hartop, 3,992,562 to Denzinger et al., 4,002,173 to Manning et al., 4,014,335 to Arnold, 4,207,893 to Michaels, and in Handbook of Common Polymers, (Scott and Roff, Eds.) Chemical Rubber Company, Cleveland, Ohio.

Hydrogels can be categorized as chemical or physical, based on the nature of the crosslinking forces that hold the hydrogel-forming molecules together. Chemical gels have stable point covalent crosslinks, while physical gels are three-dimensional networks in which polymer chains form junction zones through non-covalent interaction. Suitable hydrogel-forming compounds include hyaluronic acid. Hyaluronic acid is a natural, high-viscosity mucopolysaccharide composed of repeating disaccharide units of N-acetyl-glucosamine and D-glucuronic acid, which forms a three-dimensional network at concentrations above 1 mg/ml of water due to enlargement of the individual polymer molecules. Below a concentration of 1 mg/ml, hyaluronic acid exists as single molecules.

HA is present in most biological systems, including the umbilical cord, in vitreous humor, and in synovial fluid. The highest concentrations of HA occur in the soft connective tissues, where it is a major component of the extracellular matrix, and in the vitreous body of the eyes. It is also present in hyaline cartilage, in synovial joint fluid (the transparent viscid lubricating fluid secreted by a membrane of an articulation, bursa, or tendon sheath), and in skin tissue—both dermis and epidermis. The concentration of hyaluronic acid in the human body ranges from less than 1 μg/ml in human blood plasma to about 4 mg/ml in the umbilical cord. Hyaluronic acid plays many important roles such as lubrication of joints and regulation of water balance in tissues, and it is removed from tissues either by local degradation by lysosomal hyaluronidase, β-glucuronidase, and β-n-acetylglucosaminidase or by lymph drainage.

The terms “drug” and “pharmaceutical” are interchangeable as used herein and have their art-recognized meanings.

The phrase “pharmaceutically acceptable carrier” as used herein means a pharmaceutically acceptable material, composition, or vehicle, such as a liquid filter, diluent, excipient, solvent, or encapsulating material, involved in carrying or transporting the subject regulators from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include (1) sugars, such as lactose, glucose, and sucrose; (2) starches, such as corn starch and potato starch; (3) cellulose, and its derivatives, such as sodium carboxymethyl cellulose, ethyl cellulose, and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil, and soybean oil; (10) glycols, such as propylene, glycol; (11) polyols, such as glycerin, sorbitol, mannitol, and polyethylene glycol; (12) esters such as ethyl oleate and ethyl laurate; (13) agar; (14) buffering agents, such as magnesium hydroxide and aluminum hydroxide; (15) alginic acid; (16) pyrogen-free water; (17) isotonic saline; (18) ethyl alcohol; (19) phosphate buffer solutions; and (20) other non-toxic compatible substances employed in pharmaceutical formulations.

“Pharmaceutically acceptable salt” refers to a cationic salt formed at any acidic (e.g., hydroxamic or carboxylic acid) group, or an anionic salt formed at any basic (e.g., amino or guanidino) group. Such salts are well known in the art. See e.g., PCT Publication 87/05297, incorporated herein by reference. Such salts are made by methods known to one of ordinary skill in the art. It is recognized that the skilled artisan may prefer one salt over another for improved solubility, stability, ease of formulation, price, and the like. Determination and optimization of such salts is within the purview of the skilled artisan's practice. Pharmaceutically acceptable salts may themselves have pharmaceutical activity. Preferred anions include halides (such as chloride), sulfonates, carboxylates, phosphates, therapeutically active carboxylates, and the like.

“Physiological conditions” describe the conditions inside an organism, for example, in vivo. Physiological conditions include the acidic and basic environments of body cavities and organs, enzymatic cleavage, metabolism, and other biological processes, and preferably refer to physiological conditions in a vertebrate, such as a mammal.

The term “prodrug” is intended to encompass compounds that, under physiological conditions, are converted into the therapeutically active agents of the present invention. A common method for making a prodrug is to include selected moieties, such as esters, that are hydrolyzed under physiological conditions to convert the prodrug to an active biological moiety. In other embodiments, the prodrug is converted by an enzymatic activity of the host animal. Prodrugs are typically formed by chemical modification of a biologically active moiety. Conventional procedures for the selection and preparation of suitable prodrug derivatives are described, for example, in Design of Prodrugs, ed. H. Bundgaard, Elsevier, 1985.

In the context of referring to the codrug according to the present invention, the term “residue of a constituent moiety” means that part of a codrug that is structurally derived from a constituent moiety apart from the functional group through which the moiety is linked to another constituent moiety. For instance, where the functional group is —NH₂, and the constituent group forms an amide (—NH—CO—) bond with another constituent moiety, the residue of the constituent moiety is that part of the constituent moiety that includes the —NH—of the amide, but excluding the hydrogen (H) that is lost when the amide bond is formed. In this sense, the term “residue” as used herein is analogous to the sense of the word “residue” as used in peptide and protein chemistry to refer to a residue of an amino acid in a peptide.

By “sustained release” it is meant for purposes of the present invention that the therapeutically active medicament is released from the formulation at a controlled rate such that therapeutically beneficial levels (but below toxic levels) of the medicament are maintained over an extended period of time. Exemplary non-limiting ranges may be from about several hours to two weeks, thus, providing, for example, a two week dosage form.

The term “subject” refers to both humans and animals.

The term “symptoms” is intended to encompass any and all symptoms. Where a symptom is said to be “reduced” it is indicated that the degree of such symptom (such as the degree of joint pain or the amount of inflammatory cells in the joints) is diminished. The present invention is not limited to any particular quantitative level. Most importantly, the present invention is not limited to the complete elimination of symptoms.

The terms “method of treating or preventing”, “method of treating”, and “method of preventing” when used in connection with these diseases, disorders, or conditions mean the amelioration, prevention, or relief from the symptoms and/or effects associated with these diseases, disorders, or conditions.

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount. Prevention of an infection includes, for example, reducing the number of diagnoses of the infection in a treated population versus an untreated control population, and/or delaying the onset of symptoms of the infection in a treated population versus an untreated control population. Prevention of pain includes, for example, reducing the frequency of, or alternatively delaying, pain sensations experienced by subjects in a treated population versus an untreated control population.

The term “treating” refers to: reversing, alleviating, ameliorating, reducing, inhibiting the progress of, or preventing a disease, disorder, or condition; stabilizing a disease, disorder, or condition, for example, arresting its development; and relieving one or more symptoms of the disease, disorder, or condition, for example, causing regression of the disease, disorder, and/or condition.

The term “treatment,” means reversal, alleviation, amelioration, reduction, inhibition, prevention, stabilization, prophylaxis, relief of, or cure of a disease, disorder, or condition. Exemplary, non-limiting disease symptoms include pain and inflammation. Exemplary, non-limiting disease conditions include osteoarthritis, rheumatoid arthritis, neoplasia, microbial infection, and angiogenesis.

The phrase “therapeutically effective amount” as used herein means that amount of a compound, material, or composition comprising a compound of the present invention which is effective for producing some desired therapeutic effect at a reasonable benefit/risk ratio applicable to any medical treatment.

A “patient” or “subject” to be treated by the subject method can mean either a human or non-human animal. The patient receiving this treatment may be any animal in need, including primates, particularly humans, other mammals such as equines, cattle, swine, and sheep, poultry, and pets in general.

The term “unit” as used herein means an individual pellet, tablet, caplet, capsule, etc.

A “substitution” or “substituent” on a small organic molecule generally refers to a valency on a multivalent atom occupied by a moiety other than hydrogen, e.g., a position on a chain or ring exclusive of the member atoms of the chain or ring. Such moieties include those defined herein and others as known in the art, for example, halogen, alkyl, alkenyl, alkynyl, azide, haloalkyl, hydroxyl, carbonyl (such as carboxyl, alkoxycarbonyl, formyl, ketone, or acyl), thiocarbonyl (such as thioester, thioacetate, or thioformate), alkoxyl, phosphoryl, phosphonate, phosphinate, amine, amide, amidine, imine, cyano, nitro, azido, sulfhydryl, alkylthio, sulfate, sulfonate, sulfamoyl, sulfonamido, sulfonyl, silyl, ether, cycloalkyl, heterocyclyl, heteroalkyl, heteroalkenyl, and heteroalkynyl, heteroaralkyl, aralkyl, aryl or heteroaryl. It will be understood by those skilled in the art that certain substituents, such as aryl, heteroaryl, polycyclyl, alkoxy, alkylamino, alkyl, cycloalkyl, heterocyclyl, alkenyl, alkynyl, heteroalkyl, heteroalkenyl, and heteroalkynyl, can themselves be substituted, if appropriate. This invention is not intended to be limited in any manner by the permissible substituents of organic compounds. It will be understood that ‘substitution’ or ‘substituted with’ includes the implicit proviso that such substitution is in accordance with permitted valence of the substituted atom and the substituent, and that the substitution results in a stable compound, e.g., which does not spontaneously undergo transformation such as by rearrangement, cyclization, elimination, hydrolysis, etc.

The terms ‘amine’ and ‘amino’ are art-recognized and refer to both unsubstituted and substituted amines as well as ammonium salts, e.g., as can be represented by the general formula:

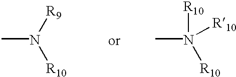

wherein R₉, R₁₀, and R′₁₀ each independently represent hydrogen or a hydrocarbon substituent, or R₉ and R₁₀ taken together with the N atom to which they are attached complete a heterocycle having from 4 to 8 atoms in the ring structure. In preferred embodiments, none of R₉, R₁₀, and R′₁₀ is acyl, e.g., R₉, R₁₀, and R′₁₀ are selected from hydrogen, alkyl, heteroalkyl, aryl, heteroaryl, carbocyclic aliphatic, and heterocyclic aliphatic. The term ‘alkylamine’ as used herein means an amine group, as defined above, having at least one substituted or unsubstituted alkyl attached thereto. Amino groups that are positively charged (e.g., R′₁₀ is present) are referred to as ‘ammonium’ groups. In amino groups other than ammonium groups, the amine is preferably basic, e.g., its conjugate acid has a pKₐ above 7.

The terms ‘amido’ and ‘amide’ are art-recognized as an amino-substituted carbonyl, such as a moiety that can be represented by the general formula:

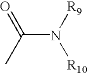

wherein R₉ and R₁₀ are as defined above. In certain embodiments, the amide will include imides. In general, when the oxygen of the above formula is replace by sulfur, the formula represents a ‘thioamide’.

The term ‘carbonyl’ is art-recognized and includes such moieties as can be represented by the general formula:

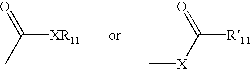

wherein X is a bond or represents an oxygen or a sulfur, and R₁₁ represents a hydrogen, hydrocarbon substituent, or a pharmaceutically acceptable salt, R₁₁′ represents a hydrogen or hydrocarbon substituent. Where X is an oxygen and R₁₁ or R₁₁′ is not hydrogen, the formula represents an ‘ester’. Where X is an oxygen, and R₁₁ is as defined above, the moiety is referred to herein as a carboxyl group, and particularly when R₁₁ is a hydrogen, the formula represents a ‘carboxylic acid’. Where X is an oxygen, and R ₁₁′ is hydrogen, the formula represents a ‘formate’. In general, where the oxygen atom of the above formula is replaced by sulfur, the formula represents a ‘thiocarbonyl’ group. Where X is a sulfur and R₁₁ or R₁₁′ is not hydrogen, the formula represents a ‘thioester.’ Where X is a sulfur and R₁₁ is hydrogen, the formula represents a ‘thiocarboxylic acid.’ Where X is a sulfur and R₁₁′ is hydrogen, the formula represents a ‘thioformate.’ On the other hand, where X is a bond, R₁₁ is not hydrogen, and the carbonyl is bound to a hydrocarbon, the above formula represents a ‘ketone’ group. Where X is a bond, R₁₁ is hydrogen, and the carbonyl is bound to a hydrocarbon, the above formula represents an ‘aldehyde’ or ‘formyl’ group.

‘Carbamate’ refers to the group having the following general structure

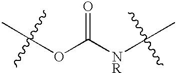

wherein R represents hydrogen or a hydrocarbon substituent.

A ‘thiocarbamate’ refers to a variant of the above group wherein the oxygen of the carbonyl is replaced by sulfur.

‘Carbonate’ refers to the group having the following general structure of

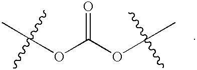

A ‘thiocarbonate’ refers to a variant of the above structure wherein the oxygen of the carbonyl is replaced by sulfur.

‘Cyclic ketal’ refers to a cyclic aliphatic group including two oxygen atoms, such as moieties having one of the following general structures:

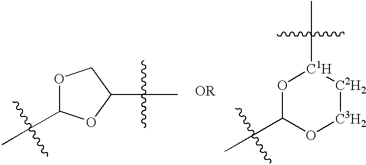

wherein substituents, such as the one depicted on C¹, could also, alternatively or additionally, be present at any other position(s) on the ring, such as on C² or C³, and/or two substituents can be present on the same position of the ring. Two carbons of the three carbons, C¹, C², and C³, together may be included in another ring structure having from 4 to 8 atoms in the ring structure.

‘Phosphate ester’ has refers to a group having the following general structure

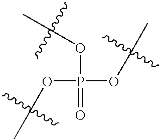

wherein each of the groups attached to the oxygens may be hydrogen, hydrocarbon, or a counterion (such as sodium) or other substituents as defined above.

A cyclic phosphate ester has the following general structure

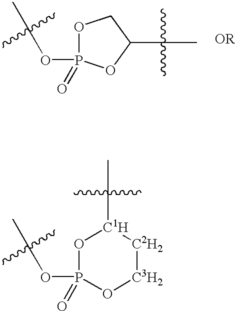

wherein substituents, such as the one depicted on C¹, could also, alternatively or additionally, be present at any other position(s) on the ring, such as on C² or C³, and/or two substituents can be present on the same position of the ring. Two carbons of the three carbons, C¹, C², and C³, together may be included in another ring structure having from 4 to 8 atoms in the ring structure.

‘Guanidino’ refers to a group having the following general structure

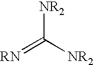

wherein each R may be, independently for each occurrence, a hydrogen or a hydrocarbon substituent. Two R's taken together may form a ring. The general structure may thus be part of one ring or a polycyclic structure.

‘Amidines’ are represented by the general formula

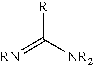

and are basic groups wherein each R may be, independently for each occurrence, a hydrogen or a hydrocarbon substituent. Two R's taken together may form a ring.

‘Hydrocarbon substituents’ are moieties that include at least one C—H bond, and include groups such as alkyl, heteroalkyl, aryl, heteroaryl, carbocyclic aliphatic, and heterocyclic aliphatic groups.

‘Heteroatom’ refers to a multivalent non-carbon atom, such as a boron, phosphorous, silicon, nitrogen, sulfur, or oxygen atom, preferably a nitrogen, sulfur, or oxygen atom. Groups containing more than one heteroatom may contain different heteroatoms.

‘Heterocyclic aliphatic ring’ is a non-aromatic saturated or unsaturated ring containing carbon and from 1 to about 4 heteroatoms in the ring, wherein no two heteroatoms are adjacent in the ring and preferably no carbon in the ring attached to a heteroatom also has a hydroxyl, amino, or thiol group attached to it. Heterocyclic aliphatic rings are monocyclic, or are fused or bridged bicyclic ring systems. Monocyclic heterocyclic aliphatic rings contain from about 4 to about 10 member atoms (carbon and heteroatoms), preferably from 4 to 7, and most preferably from 5 to 6 member atoms in the ring. Bicyclic heterocyclic aliphatic rings contain from 8 to 12 member atoms, preferably 9 or 10 member atoms in the ring. Heterocyclic aliphatic rings may be unsubstituted or substituted with from 1 to about 4 substituents on the ring. Preferred heterocyclic aliphatic ring substituents include halo, cyano, lower alkyl, heteroalkyl, haloalkyl, phenyl, phenoxy or any combination thereof. More preferred substituents include halo and haloalkyl. Heterocyclyl groups include, for example, thiophene, thianthrene, furan, pyran, isobenzofuran, chromene, xanthene, phenoxathin, pyrrole, imidazole, pyrazole, isothiazole, isoxazole, pyridine, pyrazine, pyrimidine, pyridazine, indolizine, isoindole, indole, indazole, purine, quinolizine, isoquinoline, hydantoin, oxazoline, imidazolinetrione, triazolinone, quinoline, phthalazine, naphthyridine, quinoxaline, quinazoline, quinoline, pteridine, carbazole, carboline, phenanthridine, acridine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. Preferred heterocyclic aliphatic rings include piperazyl, morpholinyl, tetrahydrofuranyl, tetrahydropyranyl and piperidyl. Heterocycles can also be polycycles.

‘Heteroalkyl’ is a saturated or unsaturated chain of carbon atoms and at least one heteroatom, wherein no two heteroatoms are adjacent. Heteroalkyl chains contain from 1 to 18 member atoms (carbon and heteroatoms) in the chain, preferably 1 to 12, more preferably 1 to 6, more preferably still 1 to 4. Heteroalkyl chains may be straight or branched. Preferred branched heteroalkyl have one or two branches, preferably one branch. Preferred heteroalkyl are saturated. Unsaturated heteroalkyl have one or more double bonds and/or one or more triple bonds. Preferred unsaturated heteroalkyl have one or two double bonds or one triple bond, more preferably one double bond. Heteroalkyl chains may be unsubstituted or substituted with from 1 to about 4 substituents unless otherwise specified. Preferred heteroalkyl are unsubstituted. Preferred heteroalkyl substituents include halo, aryl (e.g., phenyl, tolyl, alkoxyphenyl, alkoxycarbonylphenyl, halophenyl), heterocyclyl, heteroaryl. For example, alkyl chains substituted with the following substituents are heteroalkyl: alkoxy (e.g., methoxy, ethoxy, propoxy, butoxy, pentoxy), aryloxy (e.g., phenoxy, chlorophenoxy, tolyloxy, methoxyphenoxy, benzyloxy, alkoxycarbonylphenoxy, acyloxyphenoxy), acyloxy (e.g., propionyloxy, benzoyloxy, acetoxy), carbamoyloxy, carboxy, mercapto, alkylthio, acylthio, arylthio (e.g., phenylthio, chlorophenylthio, alkylphenylthio, alkoxyphenylthio, benzylthio, alkoxycarbonylphenylthio), amino (e.g., amino, mono-, and di- C1-C3 alkylamino, methylphenylamino, methylbenzylamino, C1-C3 alkylamido, carbamamido, ureido, guanidino).

A “xanthate” refers to the group having the following general structure

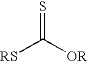

wherein R represents a hydrocarbon substituent.

II. Overview of Codrugs

Codrugs may be formed from two or more constituent moieties covalently linked together either directly or through a linking group. The covalent bonds between residues include a bonding structure such as:

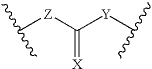

wherein Z is O, N, —CH₂—, —CH₂—O—or —CH₂—S—, Y is O, or N, and X is O or S. The rate of cleavage of the individual constituent moieties can be controlled by the type of bond, the choice of constituent moieties, and the physical form of the codrug. The lability of the selected bond type may be enzyme-specific. In some embodiments according to the present invention, the bond is selectively labile in the presence of an esterase. In other embodiments of the invention, the bond is chemically labile, e.g., to acid- or base-catalyzed hydrolysis.

In preferred embodiments according to the present invention, the linking group L does not include a sugar, a reduced sugar, a pyrophosphate, or a phosphate group.

The physiologically labile linkage may be any linkage that is labile under conditions approximating those found in physiologic fluids. The linkage may be a direct bond (for instance, ester, amide, carbamate, carbonate, cyclic ketal, thioester, thioamide, thiocarbamate, thiocarbonate, xanthate, phosphate ester, sulfonate, or a sulfamate linkage) or may be a linking group (for instance a C₁-C₁₂ dialcohol, a C-C₁₂ hydroxyalkanoic acid, a C₁-C₁₂ hydroxyalkylamine, a C₁-C₁₂ diacid, a C₁-C₁₂ aminoacid, or a C₁-C₁₂ diamine). Especially preferred linkages are direct amide, ester, carbonate, carbamate, and sulfamate linkages, and linkages via succinic acid, salicylic acid, diglycolic acid, oxa acids, oxamethylene, and halides thereof. The linkages are labile under physiologic conditions, which generally means pH of about 6 to about 8. The lability of the linkages depends upon the particular type of linkage, the precise pH and ionic strength of the physiologic fluid, and the presence or absence of enzymes that tend to catalyze hydrolysis reactions in vivo. In general, lability of the linkage in vivo is measured relative to the stability of the linkage when the codrug has not been solubilized in a physiologic fluid. Thus, while some codrugs according to the present invention may be relatively stable in some physiologic fluids, nonetheless, they are relatively vulnerable to hydrolysis in vivo (or in vitro, when dissolved in physiologic fluids, whether naturally occurring or simulated) as compared to when they are neat or dissolved in non-physiologic fluids (e.g., non-aqueous solvents such as acetone). Thus, the labile linkages are such that, when the codrug is dissolved in an aqueous solution, the reaction is driven to the hydrolysis products, which include the constituent moieties set forth above.

Codrugs for preparation of a drug delivery device according to the present invention may be synthesized in the manner illustrated in one of the synthetic schemes below. In general, where the first and second constituent moieties are to be directly linked, the first moiety is condensed with the second moiety under conditions suitable for forming a linkage that is labile under physiologic conditions. In some cases it is necessary to block some reactive groups on one, the other, or both of the moieties. Where the constituent moieties are to be covalently linked via a linker, such as oxamethylene, succinic acid, or diglycolic acid, it is advantageous to first condense the first constituent moiety with the linker. In some cases it is advantageous to perform the reaction in a suitable solvent, such as acetonitrile, in the presence of suitable catalysts, such as carbodiimides including EDCI (1-ethyl-3-(3-dimethylaminopropyl)-carbodiimide) and DCC (DCC: dicyclohexylcarbodiimide), or under conditions suitable to drive off water of condensation or other reaction products (e.g., reflux or molecular sieves), or a combination of two or more thereof. After the first constituent moiety is condensed with the linker, the combined first constituent moiety and linker may then be condensed with the second constituent moiety. Again, in some cases it is advantageous to perform the reaction in a suitable solvent, such as acetonitrile, in the presence of suitable catalysts, such as carbodiimides including EDCI and DCC, or under conditions suitable to drive off water of condensation or other reaction products (e.g., reflux or molecular sieves), or a combination of two or more thereof. Where one or more active groups have been blocked, it may be advantageous to remove the blocking groups under selective conditions, however it may also be advantageous, where the hydrolysis product of the blocking group and the blocked group is physiologically benign, to leave the active groups blocked.

The person having skill in the art will recognize that, while diacids, dialcohols, amino acids, etc., are described as being suitable linkers, other linkers are contemplated as being within the present invention. For instance, while the hydrolysis product of a codrug according to the present invention may comprise a diacid, the actual reagent used to make the linkage may be, for example, an acylhalide such as succinyl chloride. The person having skill in the art will recognize that other possible acid, alcohol, amino, sulfato, and sulfamoyl derivatives may be used as reagents to make the corresponding linkage.

Where the first and second constituent moieties are to be directly linked via a covalent bond, essentially the same process is conducted, except that in this case there is no need for a step of adding a linker. The first and second constituent moieties are merely combined under conditions suitable for forming the covalent bond. In some cases it may be desirable to block certain active groups on one, the other, or both of the constituent moieties. In some cases it may be desirable to use a suitable solvent, such as acetonitrile, a catalyst suitable to form the direct bond, such as carbodiimides including EDCI and DCC, or conditions designed to drive off water of condensation (e.g., reflux) or other reaction by-products.

The person having skill in the art will recognize that, while in most cases the first and second moieties may be directly linked in their original form, it is possible for the active groups to be derivatized to increase their reactivity. For instance, where the first moiety is an acid and the second moiety is an alcohol (i.e., has a free hydroxyl group), the first moiety may be derivatized to form the corresponding acid halide, such as an acid chloride or an acid bromide. The person having skill in the art will recognize that other possibilities exist for increasing yield, lowering production costs, improving purity, etc., of the codrug according to the present invention by using conventionally derivatized starting materials to make codrugs according to the present invention.

Exemplary reaction schemes according to the present invention are illustrated in Schemes 1-4, below. These Schemes can be generalized by substituting other therapeutic agents having at least one functional group that can form a covalent bond to another therapeutic agent having a similar or different functional group, either directly or indirectly through a pharmaceutically acceptable linker. The person of skill in the art will appreciate that these schemes also may be generalized by using other appropriate linkers.

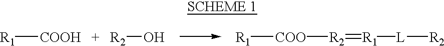

wherein L is an ester linker —COO—, and R₁ and R₂ are the residues of the first and second constituent moieties or pharmacological moieties, respectively.

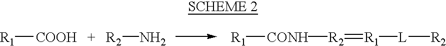

wherein L is the amide linker —CONH—, and R₁ and R₂ have the meanings given above.

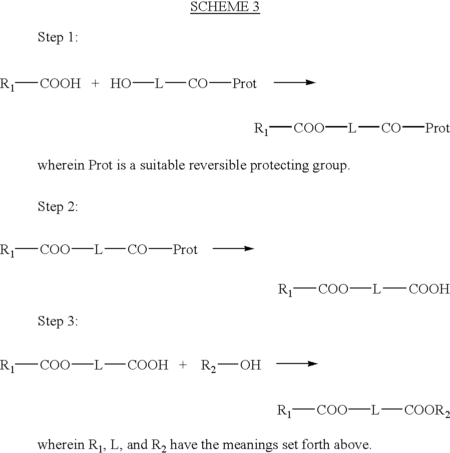

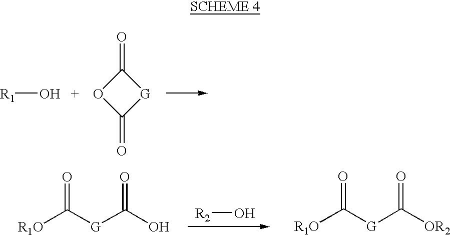

wherein R₁ and R₂ have the meanings set forth above and G is a direct bond, an C₁-C₄ alkylene, a C₂-C₄ alkenylene, a C₂-C₄ alkynylene, or a 1,2-fused ring, and G together with the anhydride group completes a cyclic anhydride. Suitable anhydrides include succinic anhydride, glutaric anhydride, maleic anhydride, diglycolic anhydride, and phthalic anhydride.

Suitable pharmaceutical compounds for use in the codrug compositions of the present invention include anti-inflammatory, analgesic, anti-angiogenic, antiviral, and antibiotic compounds. In some embodiments according to the present invention, the codrugs are water-labile, meaning that their ability to be applied intravenously or orally is severely limited due to their short half-life in aqueous solutions and biological tissues.

Suitable concentrations of codrug range from about 1 wt. % to about 99 wt. % of the pharmaceutical composition. In some embodiments of the invention, the concentration of a codrug ranges from about 5 wt. % to about 90 wt. % of the pharmaceutical composition. In certain embodiments, the concentration of a codrug ranges from about 10 wt. % to about 85 wt. %, more preferably from about 30 wt. % to about 80 wt. %, even more preferably from about 50 wt. % to about 70 wt. %, of the pharmaceutical composition.

The compositions according to the present invention may also contain one or more biologically inert or benign additives such as excipients, fillers, carriers, etc. Suitable inert or benign additives include magnesium stearate, sodium alginate, CaHPO₄, etc. Such additives may include compounds or salts that, when dissolved in water, form a buffered solution having a pH in the range of about 7.0 to about 7.6, preferably about 7.4. In some embodiments according to the present invention, such additives may constitute up from about 0 wt. % to about 50 wt. % of the pharmaceutical composition, preferably up to about 10 wt. % of the composition.

The compositions according to the present invention comprise one or more hydrogel-forming compounds, such as hyaluronic acid. Suitable hydrogel-forming compounds are those that form biodegradable gels, preferably physical gel, that are non-toxic. In some embodiments of the present invention, the hydrogel-forming compounds are physical gel-forming compounds. In certain embodiments, the hydrogel-forming compounds comprise hyaluronic acid.

Certain compositions according to the present invention substantially exclude water before they are injected into or onto living tissue. By “substantially exclude water”, it is meant that the inventive compositions contain less than about 15 wt. % water before they are injected into or onto the living biological tissue. In some embodiments according to the present invention, the inventive compositions contain less than about 12 wt. % water. In certain embodiments the inventive compositions contain less than about 10 wt. % water. However, the person skilled in the art will recognize that in some cases, crystalline forms of codrug may be used, and that such crystalline forms may contain one or more mole equivalents of water as part of the crystalline matrix. The water that is part of a crystalline form of a compound is referred to as the water of crystallization. When calculating the percent water in a mixture of hydrogel-forming compound and codrug, the water of crystallization is not included in the calculation, as the water of crystallization is properly considered in the molecular weight of the codrug.

Certain compositions according to the present invention substantially exclude water until they are hydrated prior to implantation, injection, insertion, or administration.

The compositions according to the present invention may be prepared in various physical forms, including powders, pressed-tablets, caplets, and capsules. The compositions may be prepared as powders, tablets, caplets or capsules by art-recognized methods, such as by mixing the dry powders, or by preparing a solution of the hydrogel-forming compound and a codrug in a relatively volatile solvent and then removing the solvent by evaporating, lyophilizing or spray-drying. In some embodiments according to the invention, the hydrogel-forming compound may be combined with a codrug as dry powders, which are blended.

The compositions according to the present invention may be prepared in single-dosage form, or in any dosage form, such as a partial dosage form, that the skilled artisan may conveniently administer to a patient in need of treatment with a codrug. The amount of the inventive composition in the single-dosage form will generally be chosen to be in the range of about 0.001 g to about 1.0 g, with about 0.002 g to about 0.008 g being preferred, however higher dosages, up to about 10 g may be chosen for implantation, injection, insertion, or administration into or onto certain tissues, such as the peritoneal cavity, while much lower dosages, as low as about 1 mg, may be chosen for small joints, such as knuckle or wrist joints. The proportion of codrug to hydrogel-forming compound will be chosen to optimize the release characteristics of a codrug.

Implantation, injection, insertion, or administration of the therapeutic compositions according to the invention can be accomplished by means generally known to those skilled in the art. Generally, the amount of the therapeutic composition used will depend on the specific site of the body to be treated. For some applications a single administration will often be sufficient to inhibit inflammation at the desired site. However, where continued or chronic pain is experienced (e.g., in joint inflammation), repeated applications may be used without adverse effect. Local administration is preferred via a syringe according to well established techniques, e.g., using a needle having a gauge size capable of effectively extruding the formulation while minimizing the invasiveness of the procedure.

III. Exemplary Constituent Moieties

The constituent moieties may be any biologically active moieties that possess one or more functional groups that may form hydrolyzable bonds with themselves (e.g., dimers, trimers, etc.), other biologically active moieties, or with a linkage if one is used. The constituent moieties may be, for instance, analgesic compounds such as morphine, lidocaine, benzodiazepam, tramadol, and related compounds; anti-inflammatory steroidal compounds (corticosteroids); non-steroidal anti-inflammatory compounds (NSAIDs) such as diclofenac, naproxen, ketorolac, flurbiprofen, and indomethacin; antibiotic compounds; anti-fungal compounds such as fluconazole and related compounds; antiviral compounds such as foscarnet sodium, trifluorothymidine, acyclovir, ganciclovir, dideoxyinosine (ddI), dideoxycytidine (ddC); antiproliferative compounds such as 5FU, adriamycin and related compounds; immunomodulatory compounds such as muramyl dipeptide and related compounds; cell transport/mobility impeding agents such as colchicine, vincristine, cytochalsian B, and related compounds; cytokines and peptides/proteins such as cyclosporin, insulin, growth factor or growth hormones; etc.

Exemplary antiproliferative agents include anthracyclines, vincaalkaloids, purine analogs, pyrimidine analogs, inhibitors of pyrimidine biosynthesis, and/or alkylating agents, and/or analogs, derivatives, and salts thereof. Antiproliferative compounds suitable as one or more constituent moieties in the present invention include: adriamycin, alitretinoin (9-cis-retinoic acid); amifostine; arabinosyl 5-azacytosine; arabinosyl cytosine; 5-aza-2′-deoxycytidine; 6-azacytidine; 6-azauridine; azaribine; 6-azacytidine; 5-aza-2′-deoxycytidine; bexarotene (4-[1-(5,6,7,8-tetrahydro-3,5,5,8,8-pentamethyl-2-naphthalenyl) ethenyl] benzoic acid); bleomycin; capecitabine (5′-deoxy-5-fluoro-cytidine); chlorambucil; cladribine; cytarabine; cyclocytidine; daunorubicin; 3-deazauridine; 2′-deoxy-5-fluorouridine; 5′-deoxy-5-fluorouridine; docetaxel; doxorubicin; epirubicin; estramustine; etoposide; exemestane (6-methylenandrosta-1,4-dicne-3,17-dione); fludarabine; fludarabin phosphate; fluorocytosine; 5-fluorouracil (5FU); 5-fluorouridine; 5-fluoro-2′-deoxyuridine (FUDR); gemcitabine; hydroxyurea; idarubicin; irinotecan; melphalan; methotrexate; 6-mercaptopurine; mitoxantrone; paclitaxel; pentostatin; N-phosphonoacetyl-L-aspartic acid; prednimustine; pyrazofurin; streptozocin; temozolomide; teniposide; 6-thioguanine; tomudex; topotecan; 5-trifluoromethyl-2′-deoxyuridine; valrubicin (N-trifluoroacetyladriamycin-14-valerate); vinorelbine; other modified nucleotides and nucleosides, and/or analogs, derivatives, or salts of the foregoing. Preferred antiproliferative agents are paclitaxel, docetaxel, methotrexate, 5FU, and/or analogs, derivatives, and salts thereof. Each of these antiproliferative compounds possesses one or more functional groups as defined above, and all are thus capable of being linked to one or more of the same antiproliferative compound, a different antiproliferative compound, or a different pharmaceutically active compound, having a similar or different functional group, either directly or indirectly through a pharmaceutically acceptable linker.

Suitable corticosteroids for use as one or more constituent moieties according to the present invention include: 21-acetoxypregnenolone, alclometasone, algestone, amcinonide, beclomethasone, betamethasone, budesonide, chloroprednisone, clobetasol, clobetasone,clocortolone, cloprednol, corticosterone, cortisone, cortivazol, deflazacort, desonide, desoximetasone, dexamethasone, diflorasone, diflucortolone, difuprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, hydrocortisone, loteprednol etabonate, mazipredone, medrysone, meprednisone, methylprednisolone, methylprednisolone aceponate, mometasone furoate, paramethasone, prednicarbate, prednisolone, prednisolone 25-diethylaminoacetate, prednisolone sodium phosphate, prednisone, prednival, prednylidene, rimexolone, rofleponide, tixocortol, triamcinolone, triamcinolone acetonide, triamcinolone benetonide, and triamcinolone hexacetonide, and/or analogs, derivatives, and salts thereof. Each of these corticosteroid moieties possesses one or more functional groups as defined above, and all are thus capable of being linked to one or more of the same corticosteroid, a different corticosteroid, or a different pharmaceutically active moiety.

Preferred corticosteroid moieties for preparing codrugs according to the present invention include moieties of the formula:

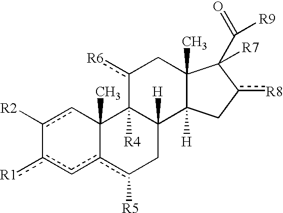

wherein R1 is ═O, —OH, or —(CH₂)₁₋₄Cl;

R2 is H, C₁₋₄alkyl, Cl, or Br;

R4 is H, F, or Cl;

R5 is H, F, Cl, CH₃, or —CHO;

R6 is H, OH, or Cl;

R7 is H, OH, CH₃, O—COCH₃, O(CO)OCH₂CH₃, O—(CO)-2-furanyl, or O—C(O)—(CH₂)₂CH_{3;}

R8 is H, CH₃, OH, ═CH₂, or together R7 and R8 form, together with the adjacent carbon atoms to which they are attached:

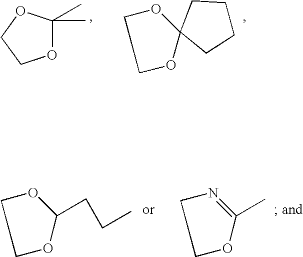

R9 is CH₃, CH₂OH, CH₂O(CO)CH₃, CH₂—O—C₁₋₄alkyl, CH₂Cl, —OCH₂Cl, —CH₂—N—(N′-methyl)piperazinyl, —CH₂—O—(CO)—CH₂—N(Et)₂, ethyl, CH₂SH, CH₂O(CO)C₁₋₄alkyl, CH₂(CO)C(2-propyl)—NH(CO)C₆H₅, or —S—CH₂—F; and

wherein the bonds indicated by [symbol] are either double or single bonds.

One skilled in the art will recognize that the class of corticosteroid compounds is a distinct class of steroids that does not include estrogens or androgens.

Illustrative examples of suitable P-lactam antibiotics include, amoxicillin, ampicillin, amylpenicillin, apalcillin, azidocillin, azlocillin, aztreonam, bacampicillin, benzylpenicillinic acid, biapenem, cefaclor, cefadroxil, cefamandole, cefatrizine, cefazedone, cefazolin, cefbuperazone, cefcapene pivoxil, cefclidin, cefdinir, cefditoren, cefepime, cefetamet, cefixime, cefmenoxime, cefmetazole, cefminox, cefodizime, cefonicid, cefoperazone, ceforanide, cefotaxime, cefotetan, cefotiam, cefoxitin, cefozopran, cefpimizole, cefpiramide, cefpirome, cefpodoxime proxetil, cefprozil, cefroxadine, cefsolodin, ceftazidime, cefteram, ceftezole, ceftibuten, ceftiofur, ceftizoxime, ceftriaxone, cefuroxime, cefuzonam, cephacetrilic acid, cephalexin, cephaloglycin, cephaloridine, cephalosporin C, cephalothin, cephamycins, cephapirinic acid, cephradine, clometocillin, cloxacillin, cyclacillin, dicloxacillin, fenbenicillin, flomoxef, floxacillin, hetacillin, imipenem, lenampicillin, loracarbef, meropenem, metampicillin, moxalactam, norcardicins (e.g., norcardicin A), oxacillin, panipenem, penicillin G, penicillin N, penicillin O, penicillin S, penicillin V, phenethicillin, piperacillin, pivampicillin, pivcefalexin, propicillin, sulbenicillin, sultamicillin, talampicillin, temocillin, ticarcillin, and tigemonam, and/or analogs, derivatives, and salts thereof. Each of the above-identified β-lactam antibiotics possesses at least one functional group capable of forming a covalent bond to at least one other pharmaceutically effective moiety having at least one functional group, either directly or via a labile linker.

Antibiotic compounds suitable as one of more constituent moieties in the present invention include: metronidazole, ciprofloxacin, amikacin, tobramycin, quinolones, etc., and/or analogs, derivatives, and salts thereof

Non-steroidal anti-inflammatory (NSAID) compounds that are suitable for R₂ possess one or more functional groups that may react with either a functional group on R₁ or a linkage to form a bond. Exemplary functional groups possessed by R₂ include hydroxy groups, amine groups, carboxylate groups (including carboxylic acids and esters), acid anhydride groups, thiol groups, sulfonyl halide groups, etc. Preferred functional groups are —OH, —NH₂, —CO₂H (including —CO₂⁻) groups, (the dashes indicating bonding to the residue of the antiproliferative compound).

NSAID compounds suitable as one or more constituent moieties in the present invention include: acetaminophen, aspirin, choline magnesium trisalicylate, diclofenac, diflunisal, etodolac, fenoprofen, flurbiprofen, ibuprofen, indomethacin, ketorolac, ketoprofen, meclofenamic acid, mefenamic acid, naproxen, nabumetone, nabumetone, oxaprozin, piroxicam, phenylbutazone, salicylic acid, sulindac, and tolmetin, and/or analogs, derivatives, and salts thereof Each of the foregoing NSAID compounds possesses at least one functional group capable of forming a direct or indirect bond to another moiety having one or more functional groups, and all are thus capable of being linked to one or more of the same NSAID, a different NSAID, or a different pharmaceutically active moiety. Preferred NSAIDs for making codrugs according to the present invention are diclofenac, flurbiprofen, naproxen, and ketoprofen. Preferred salts include sodium and potassium salts.

Suitable analgesic compounds for use as one or more constituent moieties according to the present invention include: benzodiazepam, buprenorphine, butorphanol, codeine, desmorphine, dezocine, dihydromorphine, dimepbeptanol, eptazocine, ethylmorphine, fentanyl, glafenine, hydromorphone, isoladol, ketobenidone, p-lactophetide, levorphanol, lidocaine, moptazinol, metazocin, meperidine, methadone, metopon, morphine, nalbuphine, nalmefene, nalorphine, naloxone, norlevorphanol, normorphine, oxycodone, oxymorphone, pentazocine, phenperidine, phenylramidol, propoxyphene, tramadol, and viminol, and/or analogs, derivatives, and salts thereof Each of these analgesic compounds possesses one or more functional groups as defined above, and all are analgesics capable of being linked to one or more of the same analgesic, a different analgesic, or a different pharmaceutically active moiety.

Antiandrogen compounds suitable as one of more constituent moieties in the present invention include luteinizing hormone-releasing hormone (LHRH) agonists or progestational agents, bicalutamide, bifluranol, cyproterone, flutamide, nilutamide, osaterone, oxendolone, etc., and/or analogs, derivatives, and salts thereof Each of these antiandrogen compounds possesses one or more functional groups as defined above, and all are antiandrogens capable of being linked to one or more of the same antiandrogen, a different antiandrogen, or a different pharmaceutically active moiety.

Alpha-blocker compounds suitable as one of more constituent moieties in the present invention include naftopidol and analogs of phenoxybenzamine and prazosin, and/or analogs, derivatives, and salts thereof. Each of these alpha-blocker compounds possesses one or more functional groups as defined above, and all are alpha-blockers capable of being linked to one or more of the same alpha-blocker, a different alpha-blocker, or a different pharmaceutically active moiety.

Anti-cholinergic compounds suitable as one of more constituent moieties in the present invention include biperiden, procyclidin, trihexylphenidyl hydrochloride, atropine, ipratropium bromide, oxitropium bromide, etc., and/or analogs, derivatives, and salts thereof. Each of these anti-cholinergic compounds possesses one or more functional groups as defined above, and all are anti-cholinergics capable of being linked to one or more of the same anti-cholinergic, a different anti-cholinergic, or a different pharmaceutically active moiety.

Adrenergic compounds suitable as one of more constituent moieties in the present invention include acebutolol, atenolol, betaxolol, timolol, etc., and/or analogs, derivatives, and salts thereof. Each of these adrenergic compounds possesses one or more functional groups as defined above, and all are adrenergics capable of being linked to one or more of the same adrenergic, a different adrenergic, or a different pharmaceutically active moiety.

Local anesthetic compounds suitable as one of more constituent moieties in the present invention include ambucaine, benzocaine, butamben, procaine, oxybuprocaine, tetracaine, etc., and/or analogs, derivatives, and salts thereof. Each of these local anesthetic compounds possesses one or more functional groups as defined above, and all are local anesthetics capable of being linked to one or more of the same local anesthetic, a different local anesthetic, or a different pharmaceutically active moiety.

A codrug can be administered in the form of a suspension or suspended particles in a gel that is injected, inserted, or implanted; dissolved in polymer matrix and injected, inserted, or implanted; applied topically such as a lotion, cream or spray; injected into/around bladder, prostrate, bone metastases, brain, or other tumor site or excised tumor site; incorporated into prosthetic device (e.g., plastic knee or hip) or stent; coated onto prosthetic devices, bone screws, metal plates, etc.; intraaurally administered; applied for any localized painful condition or condition that produces pain; or impregnated into gauzes, wrappings, bandages or dressings.

In particular embodiments according to the present invention, a therapeutically effective amount of a biologically active moiety, salt, or composition according to the present invention will deliver a local amount for at least 24 hours, and even more preferably may be for at least 72 hours, 100, 250, 500 or even 750 hours. In some embodiments, a local amount is delivered over at least one week, more preferably two weeks, or even more preferably at least three weeks. In certain embodiments, a local amount is delivered over at least one month, more preferably two months, and even more preferably six months.

Actual dosage levels of the active ingredients in the pharmaceutical compositions of this invention may be varied so as to obtain an amount of the active ingredient which is effective to achieve the desired therapeutic response for a particular patient and composition, without being toxic to the patient.

The selected dosage level will depend upon a variety of factors including the activity of the constituent drugs of the particular codrug employed in a drug delivery device of the present invention, or the ester, salt, or amide thereof, the time of administration, the rate of excretion of the particular codrug (and/or its constituent drugs) being employed, the duration of the treatment, other biologically active moieties, materials used in combination with the particular codrug employed, the age, species, sex, weight, condition, general health and prior medical history of the patient being treated, and like factors well known in the medical arts.

A physician or veterinarian having ordinary skill in the art can readily determine and prescribe the effective amount of the codrug required. For example, the physician or veterinarian could start doses of the codrugs of the invention employed in the drug delivery device at levels lower than that required in order to achieve the desired therapeutic effect and gradually increase the dosage until the desired effect is achieved.

IV. Exemplary Compositions

Drug delivery devices according to the present invention are suitable for implantation, for example, implantation through surgical means, needles, cannulas, catheters, etc. It may be advantageous to formulate the subject compositions in dosage unit form for ease of administration and uniformity of dosage. ‘Dosage unit form’ as used in the specification and claims herein refers to physically discrete units suitable as unitary dosages, each unit containing a predetermined quantity of active ingredient calculated to produce the desired therapeutic effect in association with the required pharmaceutical carrier. Examples of such dosage unit forms are spacers, pellets, and segregated multiples thereof.

Some embodiments of a drug delivery device according to the present invention may conveniently be presented in unit dosage forms and may be prepared by any methods well known in the art. The amount of codrug which can be combined with a material to produce a single dosage form will generally be determined from the amount of active ingredient (released from the codrug) which produces a therapeutic effect.

Some embodiments of a drug delivery device according to the present invention may be presented in single- or partial-dosage forms and hydrated prior to implantation, injection, insertion, or administration.

Methods of preparing these devices include bringing into association a codrug with a vehicle material and, optionally, one or more accessory ingredients. In some embodiments, the formulations are prepared by uniformly and intimately bringing into association a codrug with liquid vehicles, or finely divided solid vehicles, or both, and then, if necessary, shaping the product.

Codrugs may be prepared in free form, or may be prepared as salts, such as mineral acid, carboxylic acid, ammonium hydroxide or amine salts thereof. Codrugs may be prepared as amorphous or crystalline forms, and may be in the form of anhydrates or hydrates. Codrugs may be present as prodrugs, such as esters. In each of these cases, one feature is that a codrug is stable under some conditions other than physiologic conditions, and is capable of decomposing under physiologic conditions to form first and second constituent moieties, which moieties may be the same or different, as discussed above.

As set out above, certain codrugs may contain a basic functional group, such as amino or alkylamino, and are, thus, capable of forming pharmaceutically acceptable salts with pharmaceutically acceptable acids. The term “pharmaceutically acceptable salts” in this respect, refers to the relatively non-toxic, inorganic and organic acid addition salts of codrugs. These salts can be prepared in situ during the final isolation and purification of the codrugs, or by separately reacting a purified codrug of the invention in its free base form with a suitable organic or inorganic acid, and isolating the salt thus formed. Representative salts include the hydrobromide, hydrochloride, sulfate, bisulfate, formate, borate, phosphate, nitrate, acetate, valerate, oleate, palmitate, stearate, laurate, benzoate, lactate, phosphonate, tosylate, citrate, maleate, fumarate, succinate, tartrate, naphthylate, mesylate, glucoheptonate, lactobionate, and laurylsulphonate salts and the like. (See, for example, Berge et al. (1977) “Pharmaceutical Salts”, J. Pharm. Sci. 66:1-19)

The pharmaceutically acceptable salts of codrugs include the conventional nontoxic salts or quaternary ammonium salts of the codrugs, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In other cases, the codrugs may contain one or more acidic functional groups and, thus, are capable of forming pharmaceutically acceptable salts with pharmaceutically acceptable bases. The term “pharmaceutically acceptable salts” in these instances refers to the relatively non-toxic, inorganic and organic base addition salts of codrugs. These salts can likewise be prepared in situ during the final isolation and purification of the codrugs, or by separately reacting the purified codrug in its free acid form with a suitable base, such as the hydroxide, carbonate or bicarbonate of a pharmaceutically acceptable metal cation, with ammonia, or with a pharmaceutically acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like. (See, for example, Berge et al., supra)

In certain embodiments of the present invention, the pharmaceutical composition further comprises a polymer. The polymer may be non-bioerodible or bioerodible. Exemplary bioerodible polymers include polyanhydride, polylactic acid (PLA), polyglycolic acid, polyorthoester, polyalkylcyanoacrylate, and derivatives and copolymers thereof. Exemplary non-biocrodible polymers include polyurethane, polysilicone, poly(ethylene-co-vinyl acetate) (EVA), polyvinyl alcohol, and derivatives and copolymers thereof.

Other suitable polymers include poly(ethylene glycol), collagen, carbopol, hydroxypropylmethyl cellulose (“HPMC”), polypropylene, polyester, polyethylene oxide (PEO), polypropylene oxide, polycarboxylic acids, polyalkylacrylates, cellulose ethers, silicone, poly(dl-lactide-co glycolide), various Eudragrits (for example, NE30D, RS PO and RL PO), polyalkyl-alkylacrylate copolymers, polyester-polyurethane block copolymers, polyether-polyurethane block copolymers, polydioxanone, poly-(β-hydroxybutyrate), polycaprolactone, PEO-PLA copolymers, etc. The list provided above is illustrative but not limiting.

Examples of pharmaceutically acceptable antioxidants include: (1) water-soluble antioxidants, such as ascorbic acid, cysteine hydrochloride, sodium bisulfate, sodium metabisulfite, sodium sulfite, and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

Once administered, in some embodiments, the device gives a continuous supply of the codrug to the desired locus of activity without necessarily requiring additional invasive penetrations into these regions. Instead, the device may remain in the body and serve as a continuous source of the codrug to the affected area. In some embodiments, the device according to the present invention permits prolonged release of drugs over a specific period of days, weeks, months (e.g., about 3 months to about 6 months) or years (e.g., about 1 year to about 20 years, such as from about 5 years to about 10 years) until the codrug is used up.

In some embodiments, the codrugs are slowly dissolved in physiologic fluids, but upon dissolution, are relatively quickly dissociated into at least one pharmaceutically active compound. In some embodiments, the dissolution rate of the codrug is in the range of about 0.001 μg/day to about 100 μg/day. In certain embodiments, the codrugs have dissolution rates in the range of about 0.01 to about 1 μg/day. In particular embodiments, the codrugs have dissolution rates of about 0.1 μg/day.

U.S. Pat. No. 5,773,019, U.S. Pat. No. 6,001,386, and U.S. Pat. No. 6,051,576 disclose implantable controlled-release devices and drugs and are incorporated in their entireties herein by reference.

As used in regard to the low-solubility pharmaceutical codrug, the term “low-solubility” relates to the solubility of a pharmaceutical codrug in biological fluids, such as blood plasma, lymphatic fluid, peritoneal fluid, etc. In general, “low-solubility” means that the pharmaceutical codrug is only very slightly soluble in aqueous solutions having pH in the range of about 5 to about 8, and in particular to physiologic solutions, such as blood, blood plasma, etc. Some low-solubility codrugs according to the present invention will have solubilities of less than about 1 mg/ml, less than about 100 μg/ml, preferably less than about 20 μg/ml, more preferably less than about 15 μg/ml, and more preferably less than about 10 μg/ml. Solubility is measured in water at a temperature of 25° C. according to the procedures set forth in the 1995 USP, unless otherwise stated. This includes compounds which are slightly soluble (about 10 mg/ml to about 1 mg/ml), very slightly soluble (about 1 mg/ml to about 0.1 mg/ml) and practically insoluble or insoluble compounds (less than about 0.1 mg/ml).

Equivalents

Those skilled in the art will recognize, or be able to ascertain, using no more than routine experimentation, numerous equivalents to the specific biologically active moieties, methods, diluents, polymers, and salts described herein. Such equivalents are considered to be within the scope of this invention.

Exemplification

The present invention may be further appreciated upon consideration of the following illustrative and non-limiting examples.

The foregoing written description is intended to illustrate the principles of the invention, and is not intended to be limiting. One skilled in the art will readily appreciate that other embodiments are possible within the scope of the present invention, as described above and in the following claims.

All references cited herein, including patents, patent applications and non-patent literature, are explicitly incorporated herein by reference.

In the following examples, bioactive agent is used as synonymously with pharmaceutically active compound.

### EXAMPLES

The following examples are intended to illustrate an injectable drug delivery system for water-sensitive bioactive agents that are to be delivered locally, such as to a joint, and remain intact during delivery. Because hyaluronic acid is a natural component of the synovial joint fluid, it is an illustrative vehicle.

Tablets containing dry hyaluronic acid powder, the bioactive agent, and other excipients were prepared. After injecting each tablet into a joint, hyaluronic acid swells in the surrounding biological fluids and forms a physical gel with the bioactive agent incorporated therein.

The release profile of an active agent from the tablets was determined by the following in vitro studies, which reveal that, depending upon the components of the tablets, the release duration varied from about 60 to about 200 hours.

### Example 1

Sodium hyaluronate (900 mg) was combined with TC-32 (codrug of triamcinolone acetonide and 5-fluorouracil, 108 mg) and magnesium stearate (5 mg) to form a blend. Tablets of 50 mg mass and 4.5 mm diameter were hand compressed using the blend. Each tablet was then placed in a dialysis tube containing 0.5 ml of 0.1 M phosphate buffer at pH 7.4. The release study was commenced by placing each sealed dialysis tube in 100 ml of 0.1 M phosphate buffer, pH 7.4 (dialysate) at 37° C. Samples of the dialysate were taken periodically by partially or entirely replacing the dialysate with fresh buffer. The amount of TC-32 or its hydrolysis by-products (TA and 5-FU) released into the dialysate was determined by quantitative HPLC.

### Example 2

Sodium hyaluronate (200 mg) was combined with sodium alginate (80 mg), CaHPO₄ (80 mg), TC-32 (40 mg), and magnesium stearate (2.0 mg) to form a blend. Tablets of 50 mg mass and 4.5 mm diameter were hand compressed. Each tablet was then placed in a dialysis tube containing 1.0 ml of 0.1 M phosphate buffer, pH 7.4. The release study was commenced by placing each sealed dialysis tube in 100 ml of 0.1 M phosphate buffer, pH 7.4 (dialysate) at 37° C. The amount of TC-32 or its hydrolysis by-products (TA and 5-FU) released into the dialysate was determined by quantitative HPLC (see FIG. 1).

### Example 3

Sodium hyaluronate (350 mg) was combined with CaHPO₄ (150 mg), TC-32 (50 mg), and magnesium stearate (2.5 mg) and mixed to form a blend. Tablets of 50 mg mass, 4.5 mm diameter, were hand compressed using the blend. Each tablet was then placed in a dialysis tube containing 1.0 ml of 0.1 M phosphate buffer, pH 7.4. The release study was commenced by placing each sealed dialysis tube in 100 ml of 0.1 M phosphate buffer, pH 7.4 (dialysate) at 37° C. Samples were taken periodically by partially or entirely replacing the dialysate with fresh buffer. The amount of TC-32 released into the dialysate was determined by quantitative HPLC.

### Example 4

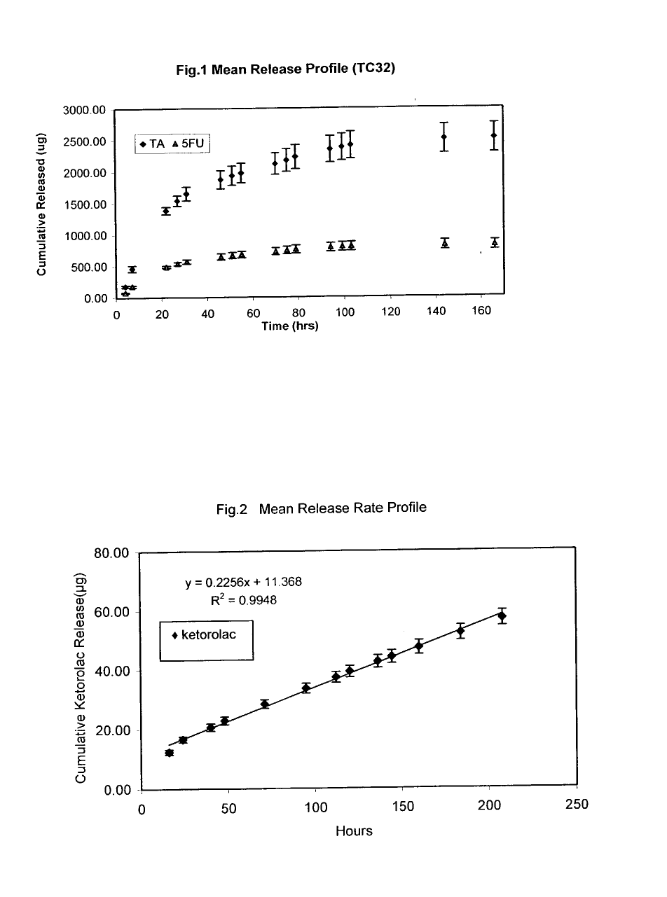

270.3 mg HA, 30.1 mg of codrug 5-TC-112.1 (codrug of ketorolac covalently linked to ketorolac via a dioxolone moiety) and 1.5 mg of magnesium stearate were mixed thoroughly to form a blend. Tablets of 25 mg, 3.0 mm diameter, were hand compressed. tablets were then placed each in a dialysis tube containing 1.0 ml 0.1 M phosphate buffer, pH 7.4. Release study was performed by placing the sealed dialysis tube in 100 ml of 0.1 M phosphate buffer, pH 7.4 at 37° C. Samples were taken periodically by partially or entirely replacing the dialysate with fresh buffer. The drug (hydrolysis products of the codrug) released in the media was determined by HPLC (see FIG. 2).

### Example 5

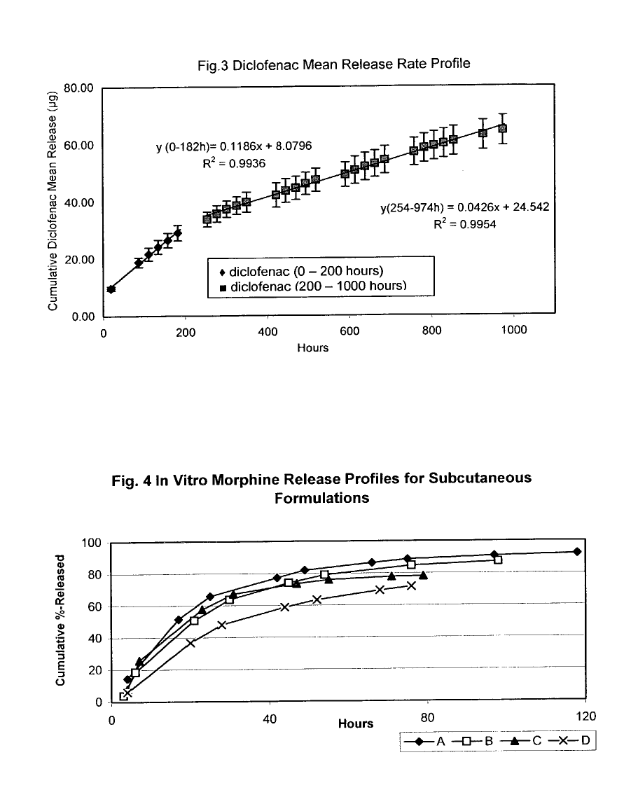

100 mg HA and 100 mg of codrug 5-TC-152.1 (codrug of diclofenac covalently linked to diclofenac via a dioxolone moiety) were mixed thoroughly to form a blend. The blend was slugged into one 1.25 cm tablet and ground into small granules, which was mixed with 1.0 mg of magnesium stearate. Pellets of 8.0 mg, 2.0 mm diameter, were hand compressed. The tablets were then placed each in a dialysis tube containing 1.0 ml 0.1 M phosphate buffer, pH 7.4. A release study was performed by placing the sealed dialysis tube in 100 ml of 0.1 M phosphate buffer, pH 7.4 at 37° C. Samples were taken periodically by partially or entirely replacing the dialysate with fresh buffer. The drug (hydrolysis products of the drug) released in the media was determined by HPLC (See FIG. 3).

### Example 6: Pellet Compositions and Preparations

Many different batches of granulation and pellets containing different compositions and with various ratios were prepared. The compositions (in weight%) for 16 selected formulations are listed in the following table.

| | | HA- | PEG | HA- | Citric | PEG | PEG | Sor- | Cyclo- |
|---|---|---|---|---|---|---|---|---|---|
| | MDM | Na | 3500 | acid | Acid | 4500 | 8000 | bitol | dexin |
| 1 | 50 | 10 | 40 | n.a | n.a | n.a | n.a | n.a | n.a |
| 2 | 50 | 20 | 30 | n.a | n.a | n.a | n.a | n.a | n.a |
| 3 | 50 | 25 | 25 | n.a | n.a | n.a | n.a | n.a | n.a |
| 4 | 50 | 30 | 20 | n.a | n.a | n.a | n.a | n.a | n.a |
| 5 | 50 | 20 | n.a | n.a | n.a | 30 | n.a | n.a | n.a |
| 6 | 55 | 25 | 10 | n.a | 10 | n.a | n.a | n.a | n.a |
| 7 | 60 | n.a | 10 | 30 | n.a | n.a | n.a | n.a | n.a |
| 8 | 60 | 30 | 10 | n.a | n.a | n.a | n.a | n.a | n.a |
| 9 | 60 | 30 | n.a | n.a | n.a | 10 | n.a | n.a | n.a |
| 10 | 60 | 30 | n.a | n.a | n.a | n.a | 10 | n.a | n.a |
| 11 | 60 | 30 | n.a | n.a | n.a | n.a | n.a | 10 | n.a |
| 12 | 60 | 30 | n.a | n.a | n.a | n.a | n.a | n.a | 10 |
| 13 | 60 | 20 | n.a | n.a | n.a | n.a | n.a | n.a | n.a |
| 14 | 60 | 40 | n.a | n.a | n.a | n.a | n.a | n.a | n.a |
| 15 | 70 | 30 | n.a | n.a | n.a | n.a | n.a | n.a | n.a |
| 16 | 75 | 25 | n.a | n.a | n.a | n.a | n.a | n.a | n.a |

MDM: Morphine-Diclofenac Maleate codrug
HA-Na: Sodium hyaluronate
HA-acid: Hyaluronic acid
PEG 3350, 4500, and 8000: Polyethylene glycol with average molecular weight of 3350, 4500, and 8000.

Generally, the compositions [morphine-diclofenac maleate (MDM), HA and/or other excipients] of individual formulation were mixed thoroughly and granulated by adding 90% ethanol followed by air-drying. The dried granules were ground to a desired particle size (visual judgment), if desired, mixed with composition not included in the granulation, followed by blending with 0.2% (weight) magnesium stearate. Using the mixture, pellets were prepared with a hand pellet press containing a 0.9 mm punch and die set. The average weight of pellet was 1.6 mg.

### Release Study

Each pellet was placed in a dialysis bag containing 1.0 ml release medium, and the bag was sealed. The bag was then immersed into 10 ml release medium. Release studies were carried out at 37° C. The early samples were taken twice daily and the later samples were taken once daily. The entire release medium was replaced following each sampling. Amounts of morphine, diclofenac, and MDM in the release medium were determined by HPLC. No intact MDM was detectable in release medium. Because of its heavy protein binding, it was difficult to quantity the amount of diclofenac in the release medium; no data for diclofenac was shown in the release profiles.

Release medium consisted of a mixture of plasma and 0.1M phosphate buffer at pH 7.4 in a 1:1 ratio was used to evaluate formulations intended for subcutaneous animal study (FIG. 4). The following table summarizes their compositions (% weight).

| Formulation | MDM | HA | PEG 3350 |
|---|---|---|---|
| A | 50 | 25 | 25 |
| B | 50 | 20 | 30 |
| C | 50 | 30 | 20 |
| D | 50 | 10 | 40 |

From the results shown in FIG. 4, it was clear that for all formulations except D, more than 75% morphine was released within two days. The Formulation C was selected for a rat pilot subcutaneous test.

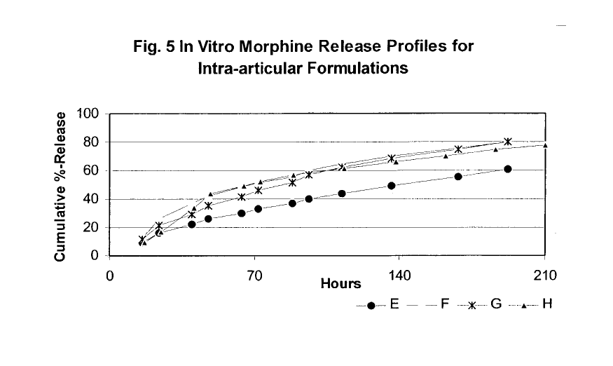

A 1 to 9 mixture of plasma and 0.1M phosphate buffer (pH 7.4) containing 2.5 mg/ml HA was used in the in vitro release studies to evaluate the formulations designed for the intra-articular (“IA”) animal studies (FIG. 5). The compositions for the formulations are shown in table underneath.

| Formulation | MDM | HA | PEG 3350 |
|---|---|---|---|
| E | 70 | 30 | NA |
| F | 60 | 30 | 10* |
| G | 50 | 30 | 20 |
| H | 60 | 30 | 10 |

*PEG 8000 was used.

Unlike plasma, synovial fluid was not commercially available but it was ascertained that concentrations of most proteins in the plasma were about 10 times higher than in synovial fluid. On other hand, synovial fluid has a higher concentration of HA (>2.5 mg/ml) while no HA is in plasma.

The release profiles in FIG. 5 showed no significant difference in release between formulations F, G and H. Greater than 74% of the total loading of morphine was detected in the release medium over 7 days. About 60% of the morphine was released from Formulation E over the same time period. The molecular weight of PEG did not affect the release profile (compare Formulations F and H). Formulation H was selected for the IA animal study.

### Example 7:

This pilot study was performed to determine the pharmacokinetics and toxicity of MDM after a single intra-articular instillation in Beagle dogs.

The study included one group of six male Beagle dogs. On Day 1 (Jul. 23, 2002) each dog was lightly tranquilized and anesthetized with a combination of atropine and medetomidine, and the area of the right stifle joint was clipped of all hair and was washed appropriately for subsequent sterile procedures. Vials containing approximately 20 mg of MDM pellets were received from the Sponsor in sterile condition. On Day 1, pellets were loaded into individual catheters using aseptic technique. Just prior to dosing, pellets were transferred into a 20 cm, 18 gauge seed implant needle. For each dog, the right hind limb was fully extended and the supra-patellar tendon was palpated. The needle was introduced into the joint and the stylet was advanced to deliver the pellets. After the stylet was removed, a 0.5 mL flush of saline was delivered through the implant needle.

Animals were monitored during the study with clinical observations daily and body weight measurements prior to dosing and necropsy. Samples were collected from each animal prior to treatment and prior to necropsy for clinical pathology analyses (hematology, serum chemistry, coagulation, and urinalysis). To further track any potential effect of MDM on coagulation profiles, samples for this parameter were also collected at 1, 4, and 24 hours after dosing. For pharmacokinetics, blood was collected from each animal at 5, 15, 30 minutes, 1, 2, 4, and 24 hours after dosing, and on Days 4, 8, and 11 (remaining animals). Samples were processed as soon as practical after collection (generally within five minutes) and transferred to the Bioanalytical Chemistry department for analysis. Two animals per time point were euthanized on Days 4, 8, and 11 and subjected to synovial fluid collection and a limited necropsy. Synovial fluid was also transferred to the Bioanalytical Chemistry department for analysis. The gross condition of the joints was described and the treated and contralateral control joints from each animal were saved in fixative for possible future analysis.

Instillation of MDM pellets was performed for each animal on Day 1 as per protocol. The actual weight of the MDM pellets administered per animal as listed on the packaging for each vial as received from the Sponsor are given below:

| Animal No. | Labeled Weight |
|---|---|
| 1001 | 17.7 mg |
| 1002 | 17.8 mg |
| 1003 | 17.9 mg |
| 1004 | 18.4 mg |
| 1005 | 18.4 mg |
| 1006 | 18.3 mg |

Clinical observations were limited to skin erythema present on the face of all dogs on Day 1. This was considered a possible reaction to the tranquilization. In addition, two dogs showed slight swelling of the right hind limb (Animal No. 1004 on Days 4 through 8; Animal No. 1005 on Days 4 through 11). There were no remarkable changes in body weight or in clinical pathology parameters (hematology, serum chemistry, coagulation, and urinalysis) as a result of treatment. Limited gross necropsy revealed findings on only two dogs. Day 8 Animal No. 1003 had a red focus and tan discoloration at the stifle joint implant site, and Day 8 Animal No. 1004 had a mottled focus on the skeletal muscle in the area of the implant site.

Analysis of plasma samples for morphine, diclofenac, and codrug concentrations revealed the following: Morphine was detectable in the dogs from as early as 5 minutes post dose to as late as Day 8 (note: some morphine concentrations present in the pretreatment samples were near the lower levels of detection and may have been due to carry-over in the assay). Diclofenac was also detectable in the plasma from about 5 to 15 minutes post dose to Day 11. Codrug was detected in the plasma of one dog (Animal No. 1002) between 15 minutes and 2 hours post does; however, the results were near the lower limits of the assay.

Diclofenac and codrug was present in the synovial fluid of both dogs at Day 4 (Animal Nos. 1001 and 1002). Morphine was also detected in the synovial fluid of Animal No. 1001. Morphine, diclofenac and codrug was detected in the synovial fluid at Day 8 of Animal No. 1003; however, only diclofenac was detected in the synovial fluid of Animal No. 1004 at Day 8. Diclofenac but no codrug was detected in the synovial fluid of Animal Nos. 1005 and 1006 at Day 11. Morphine was also detected in the synovial fluid of Animal No. 1005.

In conclusion, the instillation of MDM pellets into the stifle joint of male beagle dogs was successful in this pilot study. The procedure was well-tolerated by the dogs, and plasma and synovial fluid analysis indicated detectable levels of morphine, diclofenac and codrug.

The foregoing examples demonstrate that a composition of the present invention will release a biologically active compound, such as TC-32, gradually over time into an aqueous environment. The person having skill in the art will appreciate that this principle is generally applicable to various drugs of varying water-solubilities, various water-labilities, etc.

The person having skill in the art will recognize that the foregoing examples are presented for illustrative purposes only, to aid the person skilled in the art in practicing the claimed invention, and are not intended to be limiting. The person skilled in the art will further recognize that other embodiments are possible within the scope of the foregoing description and the following claims. All references cited herein are expressly incorporated by reference.

We claim:

1. A pharmaceutical composition comprising a codrug, or a pharmaceutically acceptable salt or prodrug thereof, in admixture with a hydrogel-forming compound, wherein the codrug comprises:
a) at least two constituent moieties, each moiety being a residue of a biologically active compound or a prodrug thereof, including a first constituent moiety and a second constituent moiety; and
b) a linkage covalently linking the at least two constituent moieties to form the codrug,
wherein the linkage is cleaved under physiological conditions to regenerate the constituent moieties.

2. The pharmaceutical composition according to claim 1, wherein the first constituent moiety is selected from analgesic compounds, anti-inflammatory steroidal compounds (corticosteroids), non-steroidal anti-inflammatory compounds (NSAIDs), antibiotic compounds, anti-fungal compounds, antiviral compounds, antiproliferative compounds, antiglaucoma compounds, immunomodulatory compounds, cell transport/mobility impeding agents, cytokines and peptides/proteins, alpha-blockers, anti-androgens, anti-cholinergic, adrenergic, puninergic, dopaminergic, local anesthetics, vanilloids, anti-angiogenic agents, nitrous oxide inhibitors, anti-apoptotic agents, macrophage activation inhibitors, and antimetabolite compounds.

3. The pharmaceutical composition according to claim 2, wherein the second constituent moiety is selected from analgesic compounds, anti-inflammatory steroidal compounds (corticosteroids), non-steroidal anti-inflammatory compounds. (NSAIDs), antibiotic compounds, anti-fungal compounds, antiviral compounds, antiproliferative compounds, antiglaucoma compounds, immunomodulatory compounds, cell transport/mobility impeding agents, cytokines and peptides/proteins, alpha-blockers, anti-androgens, anti-cholinergic, adrenergic, purinergic, dopaminergic, local anesthetics, vanilloids, anti-angiogenic agents, nitrous oxide inhibitors, anti-apoptotic agents, macrophage activation inhibitors, and antimetabolite compounds.

4. The pharmaceutical composition according to claim 1, wherein the codrug has the following structural formula:
R₁-L-(R₂)ₙ
wherein the first constituent moiety is R₁;
the second constituent moiety is R₂;
R₁ and R₂ each represent, independently, a residue of a compound selected from analgesic compounds, anti-inflammatory steroidal compounds (corticosteroids), non-steroidal anti-inflammatory compounds (NSAIDs), antibiotic compounds, anti-fungal compounds, antiviral compounds, antiproliferative compounds, antiglaucoma compounds, immunomodulatory compounds, cell transport/mobility impeding agents, cytokines and peptides/proteins, alpha-blockers, anti-androgens, anti-cholinergic, adrenergic, purinergic, dopaminergic, local anesthetics, vanilloids, anti-angiogenic agents, nitrous oxide inhibitors, anti-apoptotic agents, macrophage activation inhibitors, and antimetabolite compounds;
n is an integer of from 1 to 4; and
L is selected from a direct bond and a linking group.

5. The pharmaceutical composition according to claim 1, wherein the codrug has the following structural formula:
R₁-(L-R₂)n
wherein the first constituent moiety is R₁;
the second constituent moiety is R₂;
R₁ and R₂ each represent, independently, a residue of a compound selected from analgesic compounds, anti-inflammatory steroidal compounds (corticosteroids), non-steroidal anti-inflammatory compounds (NSAIDs), antibiotic compounds, anti-fungal compounds, antiviral compounds, antiproliferative compounds, antiglaucoma compounds, immunomodulatory compounds, cell transport/mobility impeding agents, cytokines and peptides/proteins, alpha-blockers, anti-androgens, anti-cholinergic, adrenergic, purinergic, dopaminergic, local anesthetics, vanilloids, anti-angiogenic agents, nitrous oxide inhibitors, anti-apoptotic agents, macrophage activation inhibitors, and antimetabolite compounds;
n is an integer of from 1 to 4; and
L is selected from a direct bond and a linking group.

6. The pharmaceutical composition according to claim 1, wherein the codrug has the following structural formula:
(R₁-L)ₘR₂(L₂-R₃)ₙ
wherein the first constituent moiety is R₁;
the second constituent moiety is R₂;
the third constituent moiety is R₃;
R₁, R₂, and R₃ each represent, independently, a residue of a compound selected from analgesic compounds, anti-inflammatory steroidal compounds (corticosteroids), non-steroidal anti-inflammatory compounds (NSAIDs), antibiotic compounds, anti-fungal compounds, antiviral compounds, antiproliferative compounds, antiglaucoma compounds, immunomodulatory compounds, cell transport/mobility impeding agents, cytokines and peptides/proteins, alpha-blockers, anti-androgens, anti-cholinergic, adrenergic, purinergic, dopaminergic, local anesthetics, vanilloids, anti-angiogenic agents, nitrous oxide inhibitors, anti-apoptotic agents, macrophage activation inhibitors, and antimetabolite compounds;
m is an integer of from 1 to 4;
n is an integer of from 1 to 4; and
L and L₂ are each independently selected from a direct bond and a linking group.

7. The pharmaceutical composition according to claim 4, 5, or 6, wherein R₁ is a residue of diclofenac, etodolac, ketorolac, indomethacin, salicylic acid, sulindac, tolmetin, nabumetone, piroxicam, acetaminophen, fenoprofen, flurbiprofen, ibuprofen, ketoprofen, naproxen, oxaprozin, aspirin, choline magnesium trisalicylate, diflunisal, meclofenamic acid, mefenamic acid, phenylbutazone, or salts thereof.

8. The pharmaceutical composition according to claim 4, 5, or 6, wherein R₂ is a residue of diclofenac, etodolac, ketorolac, indomethacin, salicylic acid, sulindac, tolmetin, nabumetone, piroxicam, acetaminophen, fenoprofen, flurbiprofen, ibuprofen, ketoprofen, naproxen, oxaprozin, aspirin, choline magnesium trisalicylate, diflunisal, meclofenamic acid, mefenamic acid, phenylbutazone, or salts thereof.

9. The pharmaceutical composition according to claim 4, 5, or 6, wherein R₁ is a residue of alitretinoin (9-cis-retinoic acid); amifostine; bexarotene (4-[1-(5,6,7,8-tetrahydro-3,5,5,8,8-pentamethyl-2-naphthalenyl) ethenyl] benzoic acid); bleomycin; capecitabine (5′-deoxy-5-fluoro-cytidine); chlorambucil; bleomycin; BCNU; cladribine; cytarabine; daunorubicin; docetaxel; doxorubicin; epirubicin; estramustine; etoposide; exemestane (6-methylenandrosta-1,4-diene-3,17-dione); fludarabine; 5-fluorouracil; gemcitabine; hydroxyurea; idarubicin; irinotecan; melphalan; methotrexate; mitoxantrone; paclitaxel; pentostatin; streptozocin; temozolamide; teniposide; tomudex; topotecan; valrubicin (N-trifluoroacetyladriamycin-14-valerate); or vinorelbine.

10. The pharmaceutical composition according to claim 4, 5, or 6, wherein R₂ is a residue of: 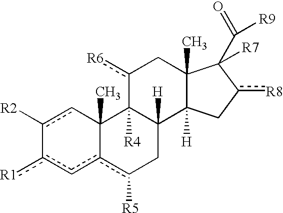
wherein R1 is ═O, —OH, or —(CH₂)₁₋₄Cl;
R2 is H, C₁₋₄alkyl, Cl, or Br;
R4 is H, F, or Cl;
R5 is H, F, Cl, CH₃, or —CHO;
R6 is H, OH, or Cl;
R7 is H, OH, CH₃, O—COCH₃, O(CO)OCH₂CH₃, O—(CO)-2-furanyl, or O—C(O)—(CH₂)₂CH₃;
R8 is H, CH₃, OH, ═CH₂, or together R7 and R8 form, together with the adjacent carbon atoms to which they are attached: 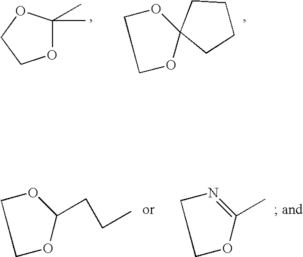
R9 is CH₃, CH₂OH, CH₂O(CO)CH₃, CH₂—O—C₁₋₄alkyl, CH₂Cl, —OCH₂Cl, —CH₂—N—(N′-methyl)piperazinyl, —CH₂—O—(CO)—CH₂—N(Et)₂, ethyl, CH₂SH, CH₂O(CO)C₁₋₄alkyl, CH₂(CO)C(2-propyl)—NH(CO)C₆H₅, or —S—CH₂—F; and
wherein the bonds indicated by [symbol] are either double or single bonds.

11. The pharmaceutical composition according to claim 4, 5, or 6, wherein R₂ is a residue of 21-acetoxypregnenolone, alclometasone, algestone, amcinonide, beclomethasone, betamethasone, budesonide, chloroprednisone, clobetasol, clocortolone, cloprednol, corticosterone, cortisone, cortivazol, deflazacort, desonide, desoximetasone, dexamethasone, diflorasone, diflucortolone, difuprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, hydrocortisone, loteprednol etabonate, mazipredone, medrysone, meprednisone, methylprednisolone, mometasone furoate, paramethasone, prednicarbate, prednisolone, prednisolone 25-diethylaminoacetate, prednisolone sodium phosphate, prednisone, prednival, prednylidene, rimexolone, tixocortol, triamcinolone, triamcinolone acetonide, triamcinolone benetonide, triamcinolone hexacetonide, and salts thereof.

12. The pharmaceutical composition according to claim 1, wherein the first constituent moiety is the same as the second constituent moiety.

13. The pharmaceutical composition according to claim 1, wherein the first constituent moiety is different from the second constituent moiety.

14. The pharmaceutical composition according to claim 1, wherein the pharmaceutical composition comprises less than 15 wt. % water.

15. The pharmaceutical composition according to claim 1 or 14, wherein the pharmaceutical composition contains less than 10 wt. % water.

16. The pharmaceutical composition according to any of claims 1-6, wherein the pharmaceutical composition comprises from about 5 wt. % to about 90 wt. % codrug.

17. The pharmaceutical composition according to any of claims 1-6, wherein the hydrogel-forming compound forms a physical gel.

18. The pharmaceutical composition according to any of claims 1-6, wherein the pharmaceutical composition is hydrated prior to implantation, injection, insertion, or administration.

19. The pharmaceutical composition according to according to any of claims 1-6, wherein said hydrogel-forming compound is hyaluronic acid or a derivative thereof.

20. The pharmaceutical composition according to claim 1, said composition is in an implantable, injectable, insertable, or otherwise administrable single-dosage form.

21. The pharmaceutical composition according to claim 1, said composition is in an implantable, injectable, insertable, or otherwise administrable partial-dosage form.

22. The pharmaceutical composition according to any of claims 1-6, 20, and 21, wherein the composition is in the form of an implantable, injectable, insertable, or otherwise administrable pellet, tablet, caplet, or capsule.

23. The pharmaceutical composition according to claim 22, wherein the composition is in the form of an implantable, injectable, insertable, or otherwise administrable pellet.

24. The pharmaceutical composition according to claim 23, wherein the pellet has a diameter from about 0.1 mm to about 5.0 mm.

25. The pharmaceutical composition according to claim 23, wherein the pellet has a length of from about 0.3 mm to about 3.0 mm.

26. The pharmaceutical composition according to claim 23, wherein the pellet is sized for administration with an 18 gauge needle.

27. The pharmaceutical composition according to claim 23, wherein the pellet weighs from about 0.5 g to about 5 g.

28. The pharmaceutical composition according to any of claims 1-6 or 16, further comprising a pharmaceutically acceptable carrier, excipient, solvent, adjuvant, additive, diluent, dispersant, or surfactant.

29. The pharmaceutical composition according to claim 28, wherein the pharmaceutically acceptable carrier comprises a biocompatible polymer.

30. The pharmaceutical composition according to claim 29, wherein the polymer is selected from collagen, carbopol, hydroxypropylmethyl cellulose (“HPMC”), polyanhydride, polylactic acid, poly(ethylene glycol) (“PEG”), and poly(ethylene-co-vinyl acetate).

31. The pharmaceutical composition according to claim 28, wherein the pharmaceutically acceptable additive is selected from sodium alginate, magnesium stearate, and CaHPO₄.

32. The pharmaceutical composition according to claim 1, which when placed in the body hydrates to release drug such that the rate of release of the drug is controlled by the dissolution of the codrug within the hydrogel.

33. The pharmaceutical composition according to claim 1, which hydrates when placed in the body and releases drug such that a diffusion coefficient of drug molecules or ions through the hydrogel is substantially the same as the diffusion coefficient of drug molecules or ions through a surrounding bodily fluid.

34. The pharmaceutical composition according to claim 1, wherein the first and second constituent moieties are directly linked through a covalent bond formed between a functional group of the first constituent moiety and a functional group of the second constituent moiety.

35. The pharmaceutical composition according to claim 1, wherein the first and second constituent moieties are linked to one another via a linking group that is covalently bonded to the first and second constituent moieties via functional groups thereon.

36. The pharmaceutical composition according to any of claims 1-6, wherein the first constituent moiety is an NSAID compound.

37. The pharmaceutical composition according to any of claims 1-6, wherein the second constituent moiety is an analgesic compound.

38. The pharmaceutical composition according to any of claims 1-6, wherein the first constituent moiety is diclofenac or ketorolac and the second constituent moiety is morphine.

39. The pharmaceutical composition according to any of claims 1-6, wherein the first constituent moiety is an antiproliferative agent and the second constituent moiety is an NSAID agent, with the proviso that the first constituent moiety is not floxuridine, and with the further proviso that when the first constituent moiety is 5-fluorouracil, the second constituent moiety is not flurbiprofen or indomethacin.

40. The pharmaceutical composition according to any of claims 1-6, wherein the first constituent moiety is an antiproliferative agent and the second constituent moiety is a corticosteroid agent, with the proviso that when the antiproliferative agent is 5-fluorouracil, the corticosteroid is not fluocinolone acetonide, triamcinolone, triamcinolone acetonide, desoximetasone, or hydrocortisone-17-butyrate, and with the further proviso that the antiproliferative agent is not a 1-β-arabinofuranosylcytosine derivative.

41. The pharmaceutical composition according to any of claims 1-6, wherein the codrug, or a pharmaceutically acceptable salt or prodrug thereof, is distributed as particles within a hydrogel-forming compound.

42. The pharmaceutical composition according to any of claims 1-6, wherein the codrug, or a pharmaceutically acceptable salt or prodrug thereof, is dissolved in a hydrogel-forming compound.

43. A method of treatment, comprising administering to a patient in need thereof a therapeutically effective amount of at least one constituent moiety in a composition comprising a codrug, or a pharmaceutically acceptable salt or prodrug thereof, in admixture with a hydrogel-forming compound, wherein the codrug comprises:
a) at least two constituent moieties, each moiety being a residue of a biologically active compound or a prodrug thereof, including a first constituent moiety and a second constituent moiety; and
b) a linkage covalently linking the at least two constituent moieties to form the codrug,
wherein the linkage is cleaved under physiological conditions to regenerate the constituent moieties.

44. The method according to claim 43, wherein the therapeutically effective amount is an amount effective to produce an analgesic, an anti-inflammatory, an antibiotic, an anti-fungal, an antiviral, and/or an antiproliferative effect in the patient.

45. A method of administering a pharmaceutical composition according to any of claims 1-6, comprising implanting the codrug composition into a synovial joint, a fibrous joint, or a cartilaginous joint, or the tissues surrounding said joint.

46. A method of administering a pharmaceutical composition according to any of claims 1-6, comprising injecting the codrug composition into a synovial joint, a fibrous joint, or a cartilaginous joint, or the tissues surrounding said joint.

47. The method according to claim 43, 45, or 46, wherein from about 5 to about 40 pellets, tablets, caplets, or capsules are administered into a synovial joint, a fibrous joint, or a cartilaginous joint, or the tissues surrounding said joint.

48. A method of administering a biologically active agent to a patient, comprising implanting, injecting, or inserting a pharmaceutical composition comprising a codrug, or a pharmaceutically acceptable salt or prodrug thereof, in admixture with a hydrogel-forming compound, for administration of at least one biologically active moiety, which codrug comprises:
a) at least two constituent moieties, each moiety being a residue of a biologically active compound or a prodrug thereof, including a first constituent moiety and a second constituent moiety; and
b) a linkage covalently linking said at least two constituent moieties to form said codrug, wherein said linkage is cleaved under physiological conditions to regenerate said constituent moieties;
wherein the composition is implanted, injected, inserted, or administered in a synovial joint, a fibrous joint, or a cartilaginous joint, or the tissues surrounding said joint.

49. A method of inhibiting cell proliferation in a patient in need of treatment, comprising implanting, injecting, or inserting a pharmaceutical composition comprising a codrug, or a pharmaceutically acceptable salt or prodrug thereof, in admixture with a hydrogel-forming compound, for administration of at least one biologically active moiety, which codrug comprises:
a) at least two constituent moieties, each moiety being a residue of a biologically active compound or a prodrug thereof, including a first constituent moiety and a second constituent moiety; and
b) a linkage covalently linking said at least two constituent moieties to form said codrug, wherein said linkage is cleaved under physiological conditions to regenerate said constituent moieties;
wherein the composition includes a therapeutically effective amount of at least one constituent moiety of a codrug, or a pharmaceutically acceptable salt thereof.

50. A method of inhibiting inflammation in a patient in need of treatment, comprising implanting, injecting, or inserting a pharmaceutical composition comprising a codrug, or a pharmaceutically acceptable salt or prodrug thereof, in admixture with a hydrogel-forming compound, for administration of at least one biologically active moiety, which codrug comprises:
a) at least two constituent moieties, each moiety being a residue of a biologically active compound or a prodrug thereof, including a first constituent moiety and a second constituent moiety; and
b) a linkage covalently linking said at least two constituent moieties to form said codrug, wherein said linkage is cleaved under physiological conditions to regenerate said constituent moieties;
wherein the composition includes a therapeutically effective amount of at least one constituent moiety of a codrug, or a pharmaceutically acceptable salt thereof.

51. The method according to claim 45 or 46, wherein the synovial joint is of a jaw, shoulder, knee, elbow, hip, ankle, wrist, finger, or toe.

52. The method according to claim 43, wherein the patient is being treated for an autoimmune disease, pain, or inflammation.

53. The method according to claim 52, wherein the autoimmune disease is rheumatoid arthritis.

54. A method of manufacturing a pharmaceutical composition, comprising providing a codrug, or a pharmaceutically acceptable salt or prodrug thereof, wherein the codrug comprises:
a) at least two constituent moieties, each moiety being a residue of a biologically active compound or a prodrug thereof, including a first constituent moiety and a second constituent moiety; and
b) a linkage covalently linking said at least two constituent moieties to form said codrug, said linkage is cleaved under physiological conditions to regenerate said constituent moieties;
and combining the codrug with a hydrogel-forming compound.

55. A method of preparing a pharmaceutical composition according to any of claims 1-6, comprising combining a powder, including a codrug, with a hydrogel-forming compound.

56. The pharmaceutical composition according to any of claims 1-6, wherein at least one constituent moiety of the codrug, taken alone, is effective for treating an autoimmune disease.

57. The pharmaceutical composition according to any of claims 1-6, wherein at least one constituent moiety of the codrug, taken alone, is effective for treating rheumatoid arthritis or osteoarthritis.

58. The pharmaceutical composition according to any of claims 1-6, wherein at least one constituent moiety of the codrug, taken alone, is effective for treating pain.

59. The pharmaceutical composition according to any of claims 1-6, wherein at least one constituent moiety of the codrug, taken alone, is effective for treating inflammation.

60. The pharmaceutical composition according to any of claims 1-6, wherein the constituent moieties are steroids.

61. The pharmaceutical composition according to any of claims 1-6 and 20, further comprising a biocompatible polymer.

62. The pharmaceutical composition according to claim 61, wherein the codrug comprises from about 5 wt. % to about 90 wt. % of the pharmaceutical composition, the hydrogel-forming compound comprises from about 10 wt. % to about 90 wt. % of the pharmaceutical composition, and the biocompatible polymer comprises from about 0 wt. % to about 50 wt. % of the pharmaceutical composition.

63. The pharmaceutical composition according to claim 62, wherein the composition substantially excludes water.

64. The pharmaceutical composition according to claim 62, wherein the biocompatible polymer is selected from collagen, carbopol, hydroxypropylmethyl cellulose (“HPMC”), polyanhydride, polylactic acid, poly(ethylene glycol), and poly(ethylene-co-vinyl acetate).

65. A pharmaceutical composition comprising poly(ethylene glycol), hyaluronic acid, and a codrug of diclofenac covalently linked to morphine.

66. The pharmaceutical composition according to claim 65, wherein a diclofenac-morphine codrug comprises from about 5 wt. % to about 90 wt. % of the pharmaceutical composition, hyaluronic acid or a derivative thereof comprises from about 10 wt. % to about 90 wt. % of the pharmaceutical composition, and the poly(ethylene glycol) comprises from about 0 wt. % to about 50 wt. % of the pharmaceutical composition.

67. The pharmaceutical composition according to claim 1, wherein the composition comprises more than one hydrogel-forming compound.

68. The pharmaceutical composition according to claim 1, wherein the composition comprises more than one polymer.

69. An injectable pellet comprising a pharmaceutical composition according to any of claims 1-6 or 65-66, wherein the pellet forms a hydrogel in vivo.

70. The method according to any of claims 43, 45, 46, 48, 49, and 50, wherein the pharmaceutical composition is hydrated prior to implantation, injection, insertion, or administration.

71. A kit comprising a pharmaceutical composition according to any of claims 1-6, 18, or 64-65, in association with instructions (written and/or pictorial) describing the use of the composition for treatment or prevention of autoimmune disease, pain, or inflammation, and optionally, warnings of possible side effects and drug-drug interactions.
